(19) United States
(12) Patent Application Publication
(10) Pub. No.: US 2009/0036369 A1
(43) Pub. Date: Feb. 5, 2009

(54) ANTI-TUMOR AGENTS COMPRISING R-SPONDINS

(75) Inventors: Makoto Kakitani, Gunma (JP); Takeshi Oshima, Gunma (JP); Kazuma Tomizuka, Gunma (JP); Kazumasa Hasegawa, Gunma (JP)

Correspondence Address: FOLEY AND LARDNER LLP;SUITE 500, 3000 K STREET NW, WASHINGTON, DC, 20007, US

(73) Assignee: Kirin Pharma Kabushiki Kaisha, Shibuya-ku, Tokyo (JP)

(21) Appl. No.: 11/996,684

(22) PCT Filed: Jul. 26, 2006
(86) PCT No.: PCT/JP2006/315255

§ 371 Date: Jan. 24, 2008

Related U.S. Application Data
(60) Provisional application No. 60/702,565, filed on Jul. 26, 2005.

(51) Int. Cl. A61K 38/16; A61K 31/7052; A61P 35/00

(52) U.S. Cl. 514/12; 514/44

(57) ABSTRACT

The present invention provides an anti-tumor agent comprising a human R-spondin including R-spondin1 (GIPF), R-spondin2, R-spondin3 or R-spondin4, or a fragment thereof which has human R-spondin activity as an active ingredient.

### TECHNICAL FIELD

The methods and compositions provided herein inhibit proliferation or migration of endothelial cells and cancer cells. The present invention relates to the field of cancer therapy. More particularly, the present invention relates to human R-spondin1 (GIPF), R-spondin2, R-spondin3, R-spondin4 and is useful in the therapy of cancer.

### BACKGROUND ART

Targeting the tumor angiogenesis is one of the effective cancer therapies. Angiogenesis refers to the sprouting, growth of small vessels, the branching, extension of existing capillaries and the assembly of endothelial cells from preexisting vessels (Folkman, J. and Shing, Y. J. Biol. Chem. 267, 10931-10934 (1992), Folkman, J. N. Engl. J. Med. 333, 1757-1763 (1995)). The initial de novo stage of vasculature formation during embryonic development is termed vasculogenesis (Risau, W. and Flamme, I. Ann. Rev. Cell Dev. Biol. 11, 73-91 (1995)). The process of angiogenesis is highly regulated through a system of naturally occurring stimulators and inhibitors. The uncontrolled angiogenesis contributes to the pathological damage associated with many diseases. Excessive angiogenesis occurs in diseases such as cancer, metastasis, diabetic blindness, diabetic retinopathy, age-related macular degeneration, atherosclerosis and inflammatory conditions such as rheumatoid arthritis and psoriasis (Ziche M. et al., Curr. Drug Targets 5, 485-493 (2004)). For example, in rheumatoid arthritis, the blood vessels in the synovial lining of the joints undergo inappropriate angiogenesis. In addition to forming new vascular networks, the endothelial cells release factors and reactive oxygen species that lead to pannus growth and cartilage destruction, and thus may actively contribute to, and help maintain, the chronically inflamed state of rheumatoid arthritis (Bodolay E. et al., J. Cell Mol. Med. 6, 357-76 (2002)). Similarly, in osteoarthritis, the activation of the chondrocytes by angiogenic-related factors may contribute to the destruction of the joint (Walsh D. A. et al., Arthritis Res. 3, 147-53 (2001)).

Angiogenesis plays a decisive role in the growth and metastasis of cancer (Zetter B. R., Ann. Rev. Med. 49, 407-24 (1998), Folkman J., Sem. Oncol. 29, 15-18 (2002)). First, angiogenesis results in the vascularization of a primary tumor, supplying necessary nutrients to the growing tumor cells. Second, the increased vascularization of the tumor provides access to the blood stream, thus enhancing the metastatic potential of the tumor. Finally, after the metastatic tumor cells have left the site of primary tumor growth, angiogenesis must occur to support the growth and expansion of the metastatic cells at the secondary site. On the contrary, insufficient angiogenesis also induce certain disease states. For example, inadequate blood vessel growth contributes to the pathology associated with coronary artery disease, stroke, and delayed wound healing (Isner J. M. and Asahara T. J., Clin. Invest. 103, 1231-1236 (1999)).

The angiogenesis stimulators of growth factors are, e.g., Angiogenin, Angiotropin, Epidermal growth factor (EGF), Fibroblast growth factor (acidic and basic) (FGF), Granulocyte colony-stimulating factor (G-CSF), Hepatocyte growth factor/scatter factor (HGF/SF), Placental growth factor (PIGF), platelet-derived endothelial cell growth factor (PD-ECGF), Platelat-derived growth factor-BB (PDGF-BB), Connective tissue growth factor (CTGF) and Vascular endothelial growth factor (VEGF); angiogenesis stimulators of proteases and protease inhibitors are, e.g., Cathepsin, Gelatinase A, Gelatinase B, Stromelysin and Urokinase-type plasminogen activator (uPA); angiogenesis stimulators of endogenous modulators are, e.g., Alpha v Beta 3 integrin, Angiopoietin-1, Erythoropoietin, Follistatin, Hypoxia, Leptin, Midkine (MK), Nitric oxide synthase (NOS), Platelet-activating factor (PAF), Pleiotropin (PTN), Prostaglandin E, CYR61 and Thrombopoietin; angiogenesis stimulators of cytokines are, e.g., Interleukin-1, Interleukin-6 and Interleukin-8, angiogenesis stimulators of signal transduction enzymes are, e.g., Thymidine phosphorylase, Farnesyl transferase and Geranylgeranyl transferase; angiogenesis stimulators of oncogenes are, e.g., c-myc, ras, c-src, v-raf and c-jun.

The angiogenesis inhibitors of growth factors are, e.g., Transforming growth factor beta (TGF-beta); angiogenesis inhibitors of proteases and protease inhibitors are, e.g., Heparinases, Plasminogen activator-inhibitor-1 (PAI-1) and Tissue inhibitor of metalloprotease (TIMP-1, TIMP-2); angiogenesis inhibitors of endogenous modulators are, e.g., Angiopoietin-2, Angiostatin, Caveolin-1, Caveolin-2, Endostatin, Fibronectin fragment, Heparin hexasaccharide fragment, Human chorionic gonadotropin (hCG), Interferon-alpha, Interferon-beta, Interferon-gamma, Interferon inducible protein (IP-10), Isoflavones, Kringle 5 (plasminogen fragment), 2-Methoxyestradiol, Placental ribonuclease inhibitor, Platelet factor-4, Prolactin (16 Kd fragment), Proliferin-related protein (PRP), Retinoids, Tetrahydrocortisol-S, Thrombospondin, Troponin-1, Vasculostatin and Vasostatin (calreticulin fragment); angiogenesis inhibitors of cytokines are, e.g., Interleukin-10 and Interleukin-12; angiogenesis inhibitors of oncogenes are, e.g., p53 and Rb.

TNF-alpha, TGF-beta, IL-4 and IL-6 are bifunctional molecules that stimulate or inhibit angiogenesis depend on the amount, the site, the microenvironment, the presence of other cytokines (Folkman, J. N. Engl. J. Med. 333, 1757-1763 (1995), Ziche M. et al., Curr. Drug Targets 5, 485-493 (2004), Ivkovic S. et al., Development 130, 2779-2791 (2003), Babic A. M., Proc. Natl. Acad. Sci. USA 95, 6355-6360 (1998)).

The main growth factors that drive angiogenesis are vascular endothelial growth factor (VEGF) and fibroblast growth factor-2 (FGF-2). VEGF promotes specifically endothelial cell migration, proliferation and the formation of a network of arterial and venous system (Ferrara N. and Davis-Smyth T., Endocrine Rev. 18, 4-25 (1997), Leung D. W. et al., Science 246, 1306-1309 (1989)). FGF-2 stimulates wider variety types of cells than VEGF, since cognate receptors of FGF-2 are expressed on fibroblasts, smooth muscle and endothelial cells (Powers C. et al., Endocr. Relat. Cancer 7, 165-197 (2000)).

Two recent discoveries of molecular pathways in angiogenesis are proangiogenic stimulation by hypoxia state via oxygen-sensing prolyl hydroxylase and hypoxia-inducible factors (HIFs) and identification of several novel extracellular angiogenic signaling pathways, that include the notch/delta, ephrin/Eph receptor, slit/roundabout, hedgehog and sprouty. The hypoxia state of tissues or tumors outgrow initiates expression of proangiogenic gene repertoires, e.g., Angiopoietin-2, FGF, HGF, TGF, IL-6, IL-8, PDGF, VEGF and VEGF receptor etc. and induces key transcription factors or HIFs (Harris A. L., Nat. Rev. Cancer 2, 38-47 (2002)).

HIF-1alpha is unstable and rapidly degrades in normal condition via the proteosome, but as oxygen tension drops below 2%, HIF-1alpha is stabilized, translocates to the nucleus, and interact with HIF-1beta to transcribe complex gene programs. HIF-1 activation leaded to increased expression of VEGF and its receptors that regulate endothelial cell proliferation and blood vessel formation (Bicknell R. and Harris A. L., Annu. Rev. Pharmacol. Toxicol. 44, 219-238 (2004), Forsythe J. A. et al., Mol. Cell. Biol. 16, 4604-4613 (1996)). Delta4 is also one of the hypoxically induced endothelial specific genes. Delta4 was absent or poorly expressed in adult tissues but showed high expression in the vasculature of xenografted human tumors and in endogenous human tumors (Mailhos C., Differentiation 69, 135-144 (2001)). EphA2 receptor tyrosine kinase was activated by VEGF through induction of ephrinA1 ligand. The blockade of EphA receptor specifically inhibited VEGF-induced angiogenesis, endothelial cell sprouting, cell survival and migration but not basic FGF induced endothelial cell survival, migration, sprouting and corneal angiogenesis (Cheng N. et al., Mol. Cancer Res. 1, 2-11 (2002)). In situ analysis had shown magic roundabout to be absent from adult tissues, except sites of active angiogenesis, but strongly expressed on the vasculature of tumors including those of the brain, bladder and colon metastasized to the liver. This expression pattern is unique among the roundabout genes and hypoxic condition induce the expression of it (Huminiecki L., Genomics 79, 547-552 (2002)). Sonic hedgehog had no effect in vitro on endothelial-cell migration or proliferation but induced the expression of three VEGF-1 isoforms and angiopoietins-1 and -2 from interstitial mesenchymal cells (Pola R. et al., Nat. Med. 7, 706-711 (2001)). Mouse sprouty protein (Sprouty-4) is a novel receptor tyrosine kinase pathway antagonist and it showed anti-angiogenesis activity (Lee S. H., J. Biol. Chem. 276, 4128-4133 (2001)).

Numerous compounds, targeting angiogenesis for tumor therapy have been identified and are now in preclinical development or in clinical trials. Exemplary compounds include the launched anti-VEGF antibody, bevacizumab that showed efficacy in restricted targets, colorectal cancer, non-small-cell lung cancer and renal-cell cancer but not showed well efficacy in metastatic prostate cancer and metastatic breast cancer (Ferrara N. et al., Nature Drug Discov. 3, 391-400 (2004)), Thalidomide is a potent teratogen and showed antiangiogenic activity in a rabbit cornea micropocket assay (D'Amato R. J. et al., Proc. Natl. Acad. Sci. U.S.A. 91, 4082-4085 (1994)), TNP-470 that is a synthetic derivative of Aspergillus fumigatus metabolite fumagillin, potently inhibited angiogenesis in vivo and the growth of endothelial cell cultures in vitro (Benjamin E. et al., Bioorg. Med. Chem. 6, 1163-1169 (1998), ABT-510 that is a TSP-1 mimetic small peptide, showed angiogenic activity through the CD36 dependent pathway (Westphal J. R. Curr. Opin. Mol. Ther. 6, 451-457 (2004)), SU-6668 that inhibited Flk-1, FGF receptor and PDGF receptor (Laird A. D. et al., Cancer Res. 60, 4152-4160 (2000)), SU-11248 that inhibited VEGF receptor 2, PDGF receptor, c-kit and liver tyrosine kinase 3 (Schueneman A. J. et al., Cancer Res. 63, 4009-4016 (2003)), Neovastat (AE-941) that inhibited VEGF receptor 2 and matrix metalloproteases (MMPs) (Beliveau R. et al., Clin. Cancer Res. 8, 1242-1250 (2002)) etc.

TSPs are a family of extracellular matrix proteins that are involved in cell-cell and cell-matrix interaction. More than five different TSPs have been known with distinct patterns of tissue distribution (Lawler J., Curr. Opin. Cell Bio. 12: 634-640 (2000), Kristin G et al., Biochemistry 41, 14329-14339 (2002)). All five members contain the type 2 repeats, the type 3 repeats and a highly conserved C-terminal domain. The type 2 repeats are similar to the epidermal growth factor repeats, the type 3 repeats comprise a contiguous set of calcium binding sites and the C-terminal domain is involved in cell binding. In addition to these domains, TSP-1 and TSP-2 contain three copies of the type 1 repeats (Bornstein P. and Sage E. H. Methods Enzymol. 245, 62-85 (1994)).

TSP-1 is a major constituent of blood platelets and that is well established molecule in the family of TSPs, stimulates vascular smooth muscle cell proliferation and migration, but it inhibits endothelial cell proliferation and migration. TSP-1 is a 420 kDa homotrimeric matricellular glycoprotein with many distinct domains. It contains a globular domain at both amino and carboxy terminus, a region of homology with procollagen, and three types of repeated sequence motifs termed thrombospondin (TSP) type1, type2 and type3 repeats (Lawler J. J. Cell Mol. Med. 6, 1-12 (2002), Margossian S. S. et al. J. Biol. Chem. 256, 7495-7500 (1981)). TSP type1 repeats was first described in 1986 and have been found in a lot of different proteins including, brain-specific angiogenesis inhibitor 1 (BAI 1), complement components (C6, C7, C8 and C9 etc.) extracellular matrix proteins like ADAMTS, mindin, axonal guidance moleluce like F-spondin, semaphorins, SCO-spondin, TRAP proteins of Plasmodium falciparum, Connective-tissue growth factor (CTGF), CYP61 and R-spondin from Xenopus, mouse and human (Lawler J. and Hynes R. O. J. Cell Biol. 103, 1635-1648 (1986), Nishimori H. et al., Oncogene. 15, 2145-2150 (1997), Jacques-Antoine H. et al., J. Biol. Chem. 264, 18041-18051 (1989), Kuno K. and Matsushima K., J. Biol. Chem. 273, 13912-13917 (1998), Higashijima S. et al., Dev. Biol. 15, 211-227 (1997), Klar A. et al., Cell 69, 95-110 (1992), Adams R. H. et al., Mech. Dev. 57, 33-45 (1996), Goncalves-Mendes N. et al., Gene. 312, 263-270 (2003), Chattopadhyay R. et al., J. Biol. Chem. 278, 25977-25981 (2003), Mercurio S. et al., Development 131, 2137-2147 (2004), Tatiana M. et al., J. Biol. Chem. 276, 21943-21950 (2001), Kazanskaya O. et al., Dev. Cell 7, 525-534 (2004), Kamata T. et al., Biochim. Biophys. Acta. 1676, 51-62 (2004)). Several proteins that posses TSP-like type 1 repeat, e. g., ADAMTS-8 and BAI 1 are angiostatic, but CTGF promotes angiogenesis. On the contrary, several type 1 repeat containing proteins have no angiogenic effects. These include complement component proteins (including C6, C7, C8 and C9), F-spondin, SCO-spondin, semapliorins 5A and 5B and several other ADAMTS proteins (Adams J. C. and Tucker R. P., Dev. Dyn. 218, 280-299 (2000)).

TSP-1 appears to function at the cell surface to bring together membrane proteins and cytokines and other soluble factors. Membrane proteins that bind TSP-1 include integrins, heparin, integrin-associated protein (CD47), CD36, proteoglycans, transforming growth factor beta (TGF-beta) and platelet-derived growth factor.

TSP type1 (properdin-like) repeat can activate TFG-beta which is involved in regulation of cell growth, axons growth, differentiation, adhesion, migration, and cell death. TSP type1 repeat is further involved in protein binding, heparin binding, cell attachment, neurite outgrowth, inhibition of tumor progression, inhibition of angiogenesis, and activation of apoptosis. An oligopeptide of RFK that lies between the first and second TSP type1 repeat has been shown to be essential for the activation of TGF-beta by TSP-1 (Schultz-Cherry S. et al., J. Biol. Chem. 270, 7304-7310 (1995), Ribeiro S. M. F. et al., J. Biol. Chem. 274, 13586-13593 (1999)). On the contrary, a hexapeptide GGWSHVW, presents in the type 1 repeats of both TSP 1 and TSP 2, binds to active TGF-beta and inhibits activation of latenet TGF-beta by TSP 1 (Schultz-Cherry S. et al., J. Biol. Chem. 270, 7304-7310 (1995)). TGF-beta has pleiotropic effects on tumor growth. At early stages of tumorigenesis, TGF-beta may act as a tumor suppressor gene (Engle S. J. et al. Cancer Res. 59, 3379-3386 (1999), Tang B. et al. Nat. Med. 4, 802-807 (1998)). TGF-beta can induce apoptosis of several different tumor cell lines (Guo Y. and Kypianou N. Cancer Res. 59, 1366-1371 (1999)). Systemic injection of the second TSP type1 repeat of TSP containing RFK peptide into B16F10 tumor bearing mice reduces the rate of tumor growth.

The effects of TSP-1 on endothelial cells include inhibition of migration and induction of apoptosis are mediated by interaction of TSP type1 repeat with CD 36 on the endothelial cell membrane. Binding of TSP-1 to CD36 receptor leads to the recruitment of the Src-related kinase, p59-fyn and to activation of p38 MAPK. The activated p38 MAPK leads to the activation of caspase-3 and to apoptosis (Jimenez B. et al. Nat. Med. 6, 41-48 (2000)). Several synthetic peptides that are similar to the partial sequence of TSP type1 repeat inhibited endothelial cell migration in vitro and angiogenesis in vivo (Tolsma S. S. et al. J. Cell. Biol. 122, 497-511 (1993), Dawson D. W. et al. Molec. Pharmacol. 55, 332-338 (1999), Iruela-Arispe M. L. et al. Circulation 100, 1423-1431 (1999)). Synthetic peptides have been used to map the anti-angiogenic activity of TSP-1. Three sequences that are adjacent to each other within the second type1 repeat have been implicated in the inhibition of angiogenesis. The synthetic peptide that contains the CSVTCG sequence was one of the first to be identified and had been shown to bind CD36 (Tolsma S. S. et al. J. Cell Biol. 122, 497-511. (1993)). Synthetic peptides that contained the CSVTCG sequence inhibited angiogenesis induced by FGF-2 or VEGF in the chick chorioalantoic membrane (Iruela-Arispe M. L. et al. Circulation 100, 1423-1431 (1999)). The second sequence WSPW that was adjacent to the first sequence bound to heparin, inhibited binding between heparin and FGF-2 and then inhibited angiogenesis induced by FGF-2 (Neng-hua G. et al., J. Biol. Chem. 267, 19349-19355 (1992), Vogel T. et al., J. Cell Biochem. 53, 74-84 (1993)). The third sequence GVITRIR that was also adjacent to the CSVTCG sequence also inhibited endothelial cell migration when the peptide was synthesized with D-isoleucine (Dawson D. W. et al. Mol. Pharmacol. 55, 332-338 (1999)). However, not every reported proteins having angiogenic or angiostatic activity contained these peptides sequence but several proteins that showed no angiogenic effect contained one or more of them.

In general, the expression of TSP decreases in tumor cells (de Fraipont F. et al. Trends Mol. Med. 7, 401-407 (2001)). Ras induces sequential activation of PI3 kinase, Rho, and POCK, leading to activation of Myc through phosphorylation. The phosphorylation fo Myc via the signal transduction pass way enables to repress TSP expression (Watnick R. S. et al. Cancer Cell 3, 219-231 (2003)). Overexpression of TSP in various types of tumor cells inhibited angiogenesis and tumor growth when these cell were implanted in immunosuppressed animals (Weinstat-Saslow D. L. et al. Cancer Res. 54, 6504-6511 (1994), Bleuel K. et al. Proc. Natl. Acad. Sci. USA 96, 2065-2070 (1999), Streit M. et al. Am. J. Pathol. 155, 441-452 (1999), Jin R. J. et al. Cancer Gene Ther. 7, 1537-1542 (2000)).

Although the 420 kDa TSP-1 is able to diminish tumor growth through its effects on the tumor vasculature, its use in human has not seriously been contemplated because of its size, difficulty in large-scale preparations, its poor pharmacokinetics and concerns about side effects that might result from its multiple other biologic functions. In order to overcome these problems, several trials have been reported. Small peptides from the preprocollagen homology region and from the properdin repeats of TSP also inhibit angiogenesis in vitro, using the same CD36-dependent pathway as the parental molecule. However, these short peptides were at least 1,000 times less active than intact TSP-1 (Tolsma S. S. et al., J. Cell Biol. 122, 497-511 (1993)). Partial amino acid substitutions from L-Amino acid to D-Amino acid and modifications of small peptides derived form a TSP-1 type 1 repeat conferred potent antiangiogenic activity, however the serum half-life (23 min) of DI-TSPa (ABT-510) in rodents after i.v. injection suggested relatively quick clearance (Dawson D. W et al., Molec. Pharmacol. 55, 332-338 (1999), Reiher F. K. et al., Int. J. Cancer 98, 682-689 (2002), Westphal J. R., Curr. Opin. Mol. Ther. 6, 451-457 (2004)). Some trials were reported to establish a recombinant adenovirus vector, expressing antiangiogenic fragment of TSP for gene therapy. Adenovirus-mediated gene therapy with an antiangiogenic fragment of TSP inhibited human leukemia xenograft growth in nude mice (Liu P. et al. Leukemia Res. 27, 701-708 (2003)). However, adenovirus-mediated gene therapy has generally some disadvantages in clinical applications, e.g., less efficient gene transfer and immune response to viral antigens (Mizuguchi H. and Hayakawa T. Hum. Gene Ther. 15, 1034-1044 (2004), Yang Y. et al. Gene Ther. 3, 137-144 (1996), Yang Y. et al. J. Virol. 70, 7209-7212 (1996)).

The mammalian family of R-spondin proteins include four independent gene products that share 40-60% amino acid sequence identity and are predicted to share substantial structural homologies. Each of four R-spondin protein family members (R-spondin1, 2, 3, 4) contains a leading signal peptide, two adjacent cystein-rich, furin-like domains, and one thrombospondin type 1 (TSP1) domain. Two furin-like and TSP1 domains are tightly conserved; specifically, the cysteine residues show strict conservation of sequence register, suggesting a common underlying structural architecture. The following C-terminal domain is of varying length but is characterized by a region of high positive charge. The published reports to date suggest that the TSP1 and C-terminal domains are dispensable for inducing β-catenin stabilization in vitro.

The first published report describing a R-spondin type protein identified hPWTSR (R-spondin3) in a fetal brain cDNA library and documented expression of the mRNA in normal placenta, lung and muscle (Chen, J. Z., et al., Mol. Biol. Rep., 29: 287-292, 2002). Subsequently, high levels of R-spondin1 mRNA expression were observed during mouse development in the roof plate/neuroepithelium boundary (2). In this study, R-spondin1 mRNA expression was significantly reduced when assessed in a Wnt1/3a double knockout background, suggesting for the first time a possible coupling of the two proteins activities (Kamata, T., et al., Biochem. Biophys. Acta, 1676: 51-62, 2004).

Further evidence for a link between R-spondins and Wnt protein activities was found with the identification of R-spondin2 in an expression screen for Xenopus modulators of the Wnt/β-catenin pathway (Kazanskaya, O., et al., Dev. Cell, 7: 525-534, 2004). In Wnt-responsive reporter assays, Xenopus R-spondin2 activated β-catenin signaling and enhanced Wnt-mediated β-catenin activation. Antisense-mediated knockdown experiments demonstrated an essential role for R-spondin2 in the embryonic development of muscle in Xenopus. The authors suggested that other R-spondin family members in addition to R-spondin2 act as soluble regulators of Wnt/β-catenin signaling (Kazanskaya, O., et al., Dev. Cell, 7: 525-534, 2004).

In addition to their role during vertebrate development, R-spondin1 has been shown to function as a potent mitogen for gastrointestinal epithelial cells (Kim, K. A., et al., Science, 309: 1256-1259, 2005). Using a functional screen of secreted proteins in transgenic mice Kim et al. recently demonstrated that human R-spondin1 expression induced a dramatic increase in proliferation of intestinal crypt epithelial cells (Kim, K. A., et al., Science, 309: 1256-1259, 2005). This proliferative effect of R-spondin1 in vivo correlates with increase activation of β-catenin and the subsequent transcriptional activation of β-catenin target genes. Moreover, these phenotypes can be recapitulated in mice injected with recombinant human R-spondin1 protein. In follow-on studies, Kim et at. have now shown that all four human R-spondin family members are capable of inducing similar effects including activation of β-catenin and proliferative effects on the gastrointestinal tract (Kim, K. A., et al., Cell Cycle, 5: 23-26, 2006). These data suggest a redundancy in the ligand activities of the R-spondin family members and given the strict conservation of predicted structural features shared by family members, it is possible that R-spondin proteins activate a common receptor or class of receptors to exert conserved biological functions.

As described above, the R-spondin family has now been established as a novel family of secreted modulator of Wnt/β-catenin signaling pathway. However, to date, there is no suggestive report of anti-angiogenic or anti-tumor activity of R-spondin proteins, even though it contains tetra peptide sequence (WSPW) and weak similarity to TSP type 1 repeat. On the contrary to the TSP-1, R-spondin1 protein preparation was well accomplished and showed in vivo high level stability. These new findings of R-spondin1 functions and characteristics showed its probability for application of cancer therapy.

### DISCLOSURE OF THE INVENTION

This description includes part or all of the contents as disclosed in the description and/or drawings of U.S. Patent Provisinal Application No. US60/702,565, which is a priority document of the present application.

The present invention encompasses an anti-tumor agent which comprises human R-spondin1 (GIPF), R-spondin2, R-spondin3 and R-spondin4 as an active ingredient.

The amino acid sequence of the full length human R-spondin1 (GIPF) is represented by SEQ ID NO: 3. The human R-spondin1 (GIPF) of the present invention includes a dominant mature form and a mature form. The amino acid sequence of the dominant mature form is represented by SEQ ID NO: 6 of the sequence listing. The mature form lacks furin cleavage sequence from the dominant mature form. The amino acid sequence of the mature form is represented by SEQ ID NO:7. The present invention also comprises a fragment of human R-spondin1 (GIPF) which has the activity of R-spondin1 (GIPF). The fragment preferably includes the fragment having a homologous region to the thrombospondin type 1 domain.

The nucleotide sequence of the human R-spondin2 is registered to GenBank as an accession number of BC036554, BC027938 or NM_{—}178565, and the nucleotide sequence of the mouse R-spondin2 is registered to GenBank as an accession number of NM_{—}172815. The nucleotide sequence of the human R-spondin3 is registered to GenBank as an accession number of NM_{—}032784 or BC022367 and the nucleotide sequence of the mouse R-spondin3 is registered as an accession number of BC103794. The nucleotide sequence of the human R-spondin4 is registered to GenBank as an accession number of NM_{—}001029871, AK122609 and the nucleotide sequence of the mouse R-spondin4 is registered to GenBank as an accession number of BC048707.

The R-spondin2 includes full length (FL) type R-spondin2 and dC type R-spondin2. The dC type R-spondin2, which was described in the report by Kazanskaya et al. (Dev. Cell, vol.7: 525-534, 2004), consists of 185 amino acids, which has the amino acid sequence consisting of 22^{nd} to 206^{th} amino acids of SEQ ID NO: 13. It lacks a region containing amino acids rich in charge at C-terminal region. It is encoded by a nucleotide sequence consisiting of 64^{th} to 621^{st} nucleotides of SEQ ID NO:12, which is corresponding to 22^{nd} to 206^{th} amino acids of the amino acid sequence of GenBank accession No. NM_{—}178565 (244 amino acids in full length). The 1^{st} to 21^{st} amino acids of SEQ ID NO:13 is a replaced signal peptide. The FL type R-spondin2 has the sequence of GenBank accession No. BC036554, BC027938 or NM_{—}178565. The present invention also comprises a fragment of human R-spondin2 which has the activity of R-spondin2. The fragment preferably includes the fragment having a homologous region to the thrombospondin type 1 domain.

The FL type R-spondin3 is a full length R-spondin3, which consists of 251 amino acids, which has the amino acid sequence consisting of 22^{nd} to 272^{nd} amino acid of SEQ ID NO: 15. It is encoded by a nucleotide sequence consisiting of 64^{th} to 819^{st} nucleotides of SEQ ID NO: 14, which is corresponding to 22^{nd} to 272^{nd} amino acids of the amino acid sequence of GenBank accession No. NM_{—}032784. The 1^{st} to 21^{st} amino acids of SEQ ID NO:15 is a replaced signal peptide. The present invention also comprises a fragment of human R-spondin3 which has the activity of R-spondin3. The fragment preferably includes the fragment having a homologous region to the thrombospondin type 1 domain.

The FL type R-spondin4 is the full length human R-spondin4 consisiting of 234 amino acids represented by SEQ ID NO: 17 and encoded by the nucletide sequence represented by SEQ ID NO:16 (nucleotide sequence from 98^{th} to 802^{nd} of the nucleotide sequence of GenBank Accession number AK12260). The present invention also comprises a fragment of human R-spondin4 which has the activity of R-spondin4. The fragment preferably includes the fragment having a homologous region to the thrombospondin type 1 domain.

A variant of R-spondin1 (GIPF), R-spondin2, R-spondin3 and R-spondin4, for example, a splice varant thereof, can be used. The human R-spondind1 (GIPF) includes a variant which has an amino acid sequence derived from the amino acid sequence represented by SEQ ID NO: 3, 6 or 7 by deletion, substitution, or addition of 1 or several amino acids, and has R-spondind1 (GIPF) activity. The number of amino acids which can be deleted, substituted or added is 1 to 10, preferably 1 to 5. The human R-spondind1 (GIPF) also includes a mutant which has an amino acid sequence having a degree of homology with the entire amino acid sequence represented by SEQ ID NO: 3, 6 or 7, such as an overall mean homology of approximately 70% or more, preferably approximately 80% or more, further preferably approximately 90% or more, and particularly preferably approximately 95% or more. Numerical values of homology described in this specification may be calculated using a homology search program known by persons skilled in the art, such as BLAST (J. Mol. Biol., 215, 403-410 (1990)) and FASTA (Methods. Enzymol., 183, 63-98 (1990)). Preferably, such numerical values are calculated using default (initial setting) parameters in BLAST or using default (initial setting) parameters in FASTA.

The present invention further encompasses an anti-tumor agent which comprises a DNA encoding human R-spondin1 (GIPF), R-spondin2, R-spondin3 or R-spondin4 as an active ingredient. The anti-tumor agent comprising the DNA encoding human R-spondin1 (GIPF), R-spondin2, R-spondin3 or R-spondin4 can be used for gene therapy. The DNA can be applied to gene thrapy by the known techniques. The DNA encoding human R-spondin1 (GIPF) has a nucleotide sequence represented by SEQ ID NO: 1 or 2. It also has a nucleotide sequence which encodes a protein having an amino acid sequence represented by SEQ ID NO: 3, 6 or 7. The variant DNA includes a DNA hybridizing under stringent conditions to the DNA having the nucleotide sequence represented by SEQ ID NO: 1 or 2, or the nucleotide sequence encoding a protein having an amino acid sequence represented by SEQ ID NO: 3, 6 or 7, and encoding a protein having human R-spondin1 (GIPF) activity. Hybridization can be carried out according to a method known in the art such as a method described in Current Protocols in Molecular Biology (edited by Frederick M. Ausubel et al., 1987)) or a method according thereto. Here, “stringent conditions” are, for example, conditions of approximately “1×SSC, 0.1% SDS, and 37° C.,” more stringent conditions of approximately “0.5×SSC, 0.1% SDS, and 42° C.,” or even more stringent conditions of approximately “0.2×SSC, 0.1% SDS, and 65° C. ”

The variant DNA also includes a nucleotide sequence that has a degree of overall mean homology with the entire nucleotide sequence of the above DNA, such as approximately 80% or more, preferably approximately 90% or more, and more preferably approximately 95% or more.

The present invention also encompasses a pharmaceutical composition comprising a R-spondin1 (GIPF), R-spondin2, R-spondin3 or R-spondin4. The composition may contain a pharmaceutically acceptable carrier and additive together. Examples of such a carrier and a pharmaceutical additive include water, pharmaceutically acceptable organic solvents, collagen, polyvinyl alcohol, polyvinylpyrrolidone, carboxy vinyl polymer, sodium carboxymethylcellulose, sodium polyacrylate, sodium alginate, water-soluble dextran, sodium carboxymethyl starch, pectin, methyl cellulose, ethyl cellulose, xanthan gum, gum arabic, casein, agar, polyethylene glycol, diglycerin, glycerin, propylene glycol, vaseline, paraffin, stearyl alcohol stearic acid, human serum albumin (HSA), mannitol, sorbitol, lactose, and surfactants that are acceptable as pharmaceutical additives. An actual additive is selected alone from the above or an appropriate combination thereof is selected depending on the dosage form of a therapeutic agent of the present invention. Such an additive is not limited to the above. For example, when the therapeutic compoaition is used in the form of a formulation for injection, it is dissolved in a solvent such as physiological saline, buffer, or a glucose solution, to which an adsorption inhibitor such as Tween80, Tween20, gelatine, or human serum albumin is added, and then the resultant can be used. Alternatively, the pharmaceutical composition may also be in a freeze-dried dosage form, so that it can be dissolved and reshaped before use. As an excipient for freeze-drying, for example, sugar alcohols such as mannitol and glucose and sugars can be used. A pharmaceutical composition of the present invention is generally administered via a parenteral route of administration, such as injection (e.g., subcutaneous injection, intravenous injection, intramuscular injection, or intraperitoneal injection), transdermal administration, transmucosal administration, transnasal administration, or transpulmonary administration. Oral administration is also possible. When the pharmaceutical composition of the present invention is administered to a patient, the effective dosage per administration is selected from the range between 20 ng and 200 mg per kg of body weight. Alternatively, a dosage of 0.001 to 10000 mg/body weight, preferably 0.005 to 2000 mg/body weight, and more preferably 0.01 to 1000 mg/body weight per patient can be selected. However, the dosage of the pharmaceutical composition of the present invention is not limited to these dosages.

The anti-tumor agent and the pharmaceutical composition of the present invention can be used for treatment of or prophylaxis against various tumors. The tumor includes colon cancer, colorectal cancer, lung cancer, breast cancer, brain tumor, malignant melanoma, renal cell carcinoma, bladder cancer, leukemia, lymphomas, T cell lymphomas, multiple myeloma, gastric cancer, pancreas cancer, cervical cancer, endometrial carcinoma, ovarian cancer, esophageal cancer, liver cancer, head and neck squamous cell carcinoma, cutaneous cancer, urinary tract carcinoma, prostate cancer, choriocarcinoma, pharyngeal cancer, laryngeal cancer, thecomatosis, androblastoma, endometrium hyperplasy, endometriosis, embryoma, fibrosarcoma, Kaposi's sarcoma, hemangioma, cavernous hemangioma, angioblastoma, retinoblastoma, astrocytoma, neurofibroma, oligodendroglioma, medulloblastoma, ganglioneuroblastoma, glioma, rhabdomyosarcoma, hamartoblastoma, osteogenic sarcoma, leiomyosarcoma, thyroid sarcoma and Wilms tumor.

### BRIEF DESCRIPTION OF THE DRAWINGS

FIG. 1 is a multiple alignment of TSP-1 type 1 repeat regions between human R-Spondin 1 (GIPF) and Thrombospondin 1 (TSP1).

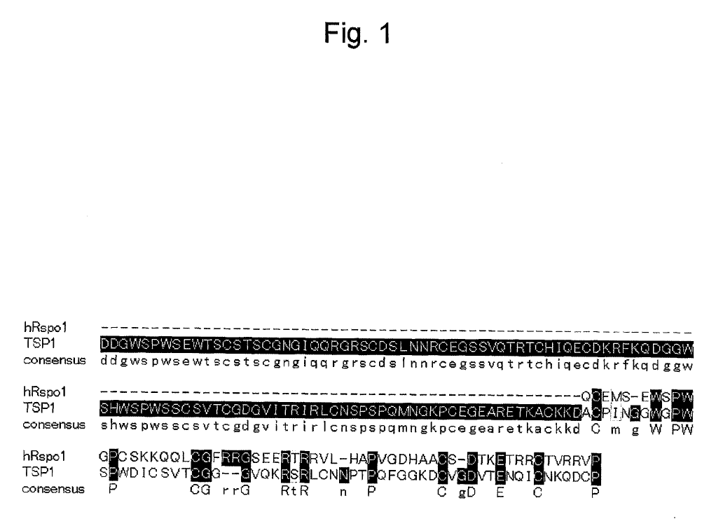

FIG. 2A is a diagram showing the effect of NaCl and Arg on the stability of the R-Spondin1 (GIPF) protein at pH7.

FIG. 2B is a diagram showing the solubility of purified protein in PBS.

FIG. 3A is a diagram showing the stability of a recombinant R-Spondin1 (GIPF) in blood.

FIG. 3B is a diagram showing the half-life of R-Spondin1 (GIPF) in serum.

FIG. 4 is a diagram showing the construct of pcmv R-Spondin1 (GIPF)-IRES-GFP.

FIG. 5 is a diagram showing the construct of pcmvEOP-IRES-GFP.

FIG. 6 is a diagram showing the results of survival curve of cell transferred mice in each group. SCa group si A-2GH GIPF expressing NIH3T3 cell transferred group, SCb group is A-5GH R-Spondin1 (GIPF) expressing NIH3T3 cell transferred group, SCc group is D-3GH human EPO expressing NIH3T3 cell transferred group, SCd group is wild-type NIH3T3 cell transferred group and Sce group is EMEM injected group as control.

FIG. 7 is a photograph showing tumor development in cell transferred mice in each group. Each group is the same with the group described in FIG. 6.

FIG. 8 is a photograph showing tumor development in cell transferred mice in each group. SCa group is A-2GH R-Spondin1 (GIPF) expressing NIH3T3 cell transferred group, SCc group is D-3GH human EPO expressing NIH3T3 cell transferred group and SCd group is wild-type N1H3T3 cell transferred group.

FIG. 9A is a diagram showing the results of measuring the Sw620 tumor size in mice when R-Spondin1 (GIPF) were administered.

FIG. 9B is a diagram showing the results of measuring the COLO205 tumor size when R-Spondin1 (GIPF) were administered.

FIG. 9C is a diagram showing the results of measuring the HT29 tumor size when R-Spondin1 (GIPF) were administered.

FIG. 10A is a graph showing the results of the effect of R-Spondin1 (GIPF) on the proliferation of normal human endotherial cells (HUVECs).

FIG. 10B is a graph showing the results of the effect of R-Spondin1 (GIPF) on the proliferation of normal human endotherial cells (HMVECs).

FIG. 11 is a graph showing the results of the effect of R-Spondin1 (GIPF) on the migration of normal human endothelial cells (HMVECs).

### BEST MODE FOR CARRYING OUT THE INVENTION

### Example 1

### Expression Vectors Encoding GIPF and V5His6-Tagged GIPF

The cDNA encoding GIPF (SEQ ID NO: 1) was cloned into pcDNA/Intron vector using KpnI and XbaI sites to generate wild type and carboxy-terminal V5His6-tagged GIPF (SEQ ID NO: 4). The mammalian expression vector pcDNA/Intron was obtained by genetically modifying the pcDNA3.1TOPO vector (Invitrogene Inc., Carlsbad, Calif.) by introducing an engineered chimeric intron derived from the pCI mammalian expression vector (Promega, Madison, Wis.). pCI was digested with BGlII and KpnI, and the intron sequence was cloned into pcDNA3.1, which had been digested with BglII and KpnI. The GIPF ORF of SEQ ID NO: 1 (SEQ ID NO: 2) was first cloned into pcDNA3.1/V5His-TOPO (Invitrogen) by PCR using the following forward 5′ CACCATGCGGCTTGGGCTGTCTC 3′ (SEQ ID NO: 8) reverse 5′ GGCAGGCCCTGCAGATGTGAGTG 3′ (SEQ ID NO: 9), and the KpnI-XbaI insert from pcDNA 3.1/V5His-TOPO that contains the entire GIPF ORF was ligated into the modified pcDNA/Intron vector to generate pcDNA/Intron construct.

### Example 2

### Purification of Recombinant GIPF

### A. Expression and Purification GIPFt in Eukaryotic Cells:

V5-His-tagged GIPF (GIPFt) (SEQ ID NO: 4) was expressed in HEK293 and CHO cells and purified as follows:

A stable cell culture of HEK293 cells that had been transfected with the GIPF pcDNA/Intron construct comprising the DNA encoding the V5-His-tagged GIPF polypeptide (SEQ ID NO: 4) was grown in serum free 293 free-style media (GIBCO). A suspension culture was seeded at cell density of 1 million cells/ml, and harvested after 4-6 days. The level of the V5-His-tagged GIPF that had been secreted into the culture medium was assayed by ELISA.

A stable cell culture of CHO cells that had been transformed with a pDEF 2S vector comprising nucleotide sequence that encodes a V5-His tagged GIPF (SEQ ID NO: 4) was grown in serum free EX-CELL302 media (JRH). The expression vector contains DNA sequence that encodes DHFR, which allows for positive selection and amplification in the presence of methotrexate (MTX). The level of the V5-His-tagged GIPF that had been secreted into the culture medium was assayed by ELISA.

The media containing the secreted GIPF protein was harvested and frozen at −80° C. The media was thawed at 4° C., and protease inhibitors, EDTA and Pefabloc (Roche, Basel, Switzerland) were added at a final concentration of 1 mM each to prevent degradation of GIPF. The media were filtered through a 0.22 μm PES filter (Corning), and concentrated 10-fold using TFF system (Pall Filtron) with a 10 kDa molecular weight cut-off membrane, The buffers of the concentrated media were exchanged with 20 mM sodium phosphate, 0.5M NaCl, pH 7. The addition of 0.5 M NaCl in the phosphate buffer is crucial to keep fill solubility of V5-His tagged GIPF at pH 7 during purification. Following utrafiltration and diafiltration, a mammalian protease inhibitor cocktail (Sigma) was added to a final dilution of 1:500 (v/v).

A HiTrap Ni²⁺-chelating affinity column (Pharmacia) was equilibrated with 20 mM sodium phosphate, pH 7, 0.5 M NaCl. The buffer-exchanged media was filtered with 0.22 μm PES filter and loaded onto Ni²⁺-chelating affinity column. The Ni²⁺ Column was washed with 10 column volumes (CV) of 20 mM imidazole for 10 Column Volume and protein was eluted with a gradient of 20 mM to 300 mM imidazole over 35 CV. The fractions were analyzed by SDS-PAGE and Western blot. Fractions containing V5-His tagged GIPF were analyzed and pooled to yield a GIPF protein solution that was between 75-80% pure.

The buffer containing the GIPF protein isolated using the Ni²⁺ column was exchanged with 20 mM sodium phosphate, 0.3 M Arginine, pH 7 to remove the NaCl. NaCl was replaced with 0.3 M Arg in the phosphate buffer to maintain full solubility of V5-His tagged GIPF protein during the subsequent purification steps. The GIPF protein isolated using the Ni²⁺ column was loaded onto a SP Sepharose high performance cation exchange column (Pharmacia, Piscataway, N.J.) that had been equilibrated with 20 mM sodium phosphate, 0.3 M Arginine, pH 7. The column was washed with 0.1 M NaCl for 8 CV, and eluted with a gradient of 0.1 M to 1 M NaCl over 30 CV. Fractions containing V5-His tagged GIPF were pooled to yield a protein solution that was between 90-95% pure.

The buffer of the pooled fractions was exchanged with 20 mM sodium phosphate, pH 7, 0.15 M NaCl, the protein was concentrated to 1 or 2 mg/mL, and passed through a sterile 0.22 μm filter. The pure GIPF preparation was stored at −80° C.

The protein yield obtained at the end of ach purification step was analyzed and quantified by ELISA, protein Bradford assay and HPLC. The percent recovery of GIPFt protein was determined at every step of the purification process, and is shown in Table 1 below.

TABLE 1

| Steps | Step Recovery | Overall Recovery |
|---|---|---|
| Media Concentration/Diafiltration | 100% | 100% |
| Ni-chelating Affinity | 65% | 65% |
| SP Cation Exchange | 80% | 52% |
| Final Formulation and filter | 95% | 49% |

SDS-PAGE analysis of the purified GIPF protein was performed under reducing and non-reducing conditions, and showed that the V5-His tagged GIPF protein derived from both CHO and 293 cells exists as a monomer. GIPF protein is glycosylated and migrates on SDS-PAGE under non-reducing conditions with molecular weight (MW) of approximately 42 kDa. There is slight difference in the MW of the GIPF protein purified from CHO cells and that purified from HEK293 cells. This difference may be explained by the extent to which GIPF is glycosylated in different cell types. N-terminal sequence analysis showed that HEK293 cells produced two forms of the polypeptide: the dominant mature form (SEQ ID NO: 6) which corresponds to the GIPF protein of SEQ ID NO: 3 that lacks the signal sequence, and the mature form (SEQ ID NO: 7), which corresponds to the GIPF protein of SEQ ID NO: 3 that lacks both the signal peptide and the furin cleavage sequence. The two forms separated well on the SP column, and were expressed at a ratio of mature to dominant mature forms of approximately 1:2.

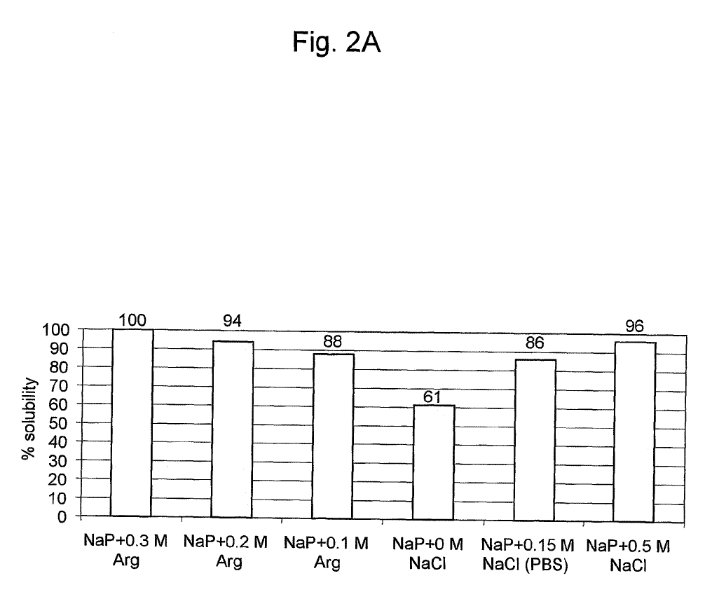

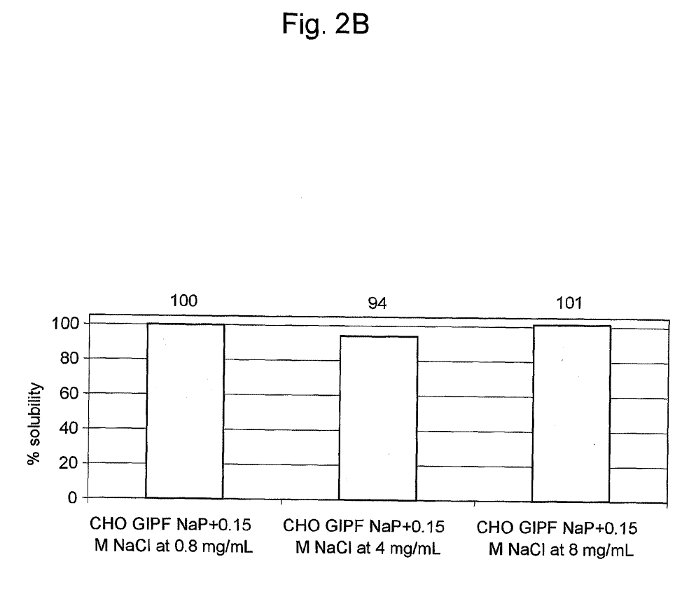

The effect of NaCl and Arginine (Arg) on the solubility of the GIPF protein at pH 7 was determined, and is shown in FIG. 2 A. It was determined that in the absence of 0.3M Arg a 50% loss of protein was incurred during the purification. FIG. 2 B shows the solubility of purified protein in PBS (20 mM sodium phosphate, 0.15 M NaCl, pH 7). GIPF protein remains in solution at concentrations of up to 8 mg/mL at 4° C., pH7, for 7 days.

In summary, the purification of V5-His-tagged GIPF from cultures of HEK293 or CHO cells was performed by 1) concentrating and diafiltering the GIPF protein present in the culture media, 2) performing Ni²⁺-chelating affinity chromatography, and 3) SP cation exchange chromatography. The purification process yields a GIPF protein that is >90% pure. The overall recovery of the current purification process is approximately 50%. Addition of 0.5 M NaCl to the buffer during the purification process of media diafiltration and Ni column is crucial to keep GIPF fully soluble at pH 7. For binding GIPF onto the SP column, NaCl was removed, and 0.3 M Arg was added to maintain high solubility and increase protein recovery. The addition of 0.5 M NaCl and 0.3 Arg during the first and second purification steps showed to increase the overall recovery by at least from 25% to 50%.

### B. Expression and Purification of GIPFwt in Eukaryotic Cells:

A stable cell culture of HEK293 cells that had been transfected with the pcDNA/Intron vector comprising the DNA (SEQ ID NO: 2) encoding the full-length GIPF polypeptide (GIPFwt) (SEQ ID NO: 3) was adapted to grow in suspension and grown in serum-free 293 free-style medium (GIBCO) in the presence of 25 μg/ml geneticin.

Cell culture growth in spinner: For small-scale production in spinners, an aliquot of a frozen stock of cells was grown and expanded in 293 free-style media with addition of 0.5% Fetal Bovine Serum (FBS). Cells were seeded and expanded in spinners at cell density of 0.3-0.5 million/mL for each passage. When enough cells are accumulated and cell density reaches 1 million cells/mL for production, the media was exchanged with serum-free 293 free-style media to remove 0.5% FBS, and harvested after 6 days. The initial cell viability was between 80-90% and it decreased to 30% at the time of harvest. The level of GIFPwt that had been secreted into the culture medium was assayed by ELISA and western. Growth of GIPFwt in the spinners yielded 1.2-1.5 mg/l.

Cell Culture Growth in Bioreactors-Fed-batch mode was used for large-scale production in bioreactors. A serum-free adapted suspension culture of HEK293 cells was seeded at cell density of 0.2-0.4 million/ml when passage of cells. Cells were grown in serum free 293 free-style medium and expanded from 50-500 ml shake flasks to 20-50 stir tanks for inoculation of a 2001 and 5001 bioreactor. When enough cells were accumulated, the cells were inoculated into a bioreactor at a density of 0.2-0.4 million cells/ml. When the cell density reached 1 million cells/ml, vitamins and MEM amino acids (GIBCO) were added to boost and support the growth. Cells were harvested from the bioreactor after 6-7 days when the cell viability had decreased to 25-30%. The level of GIPFwt that had been secreted into the culture medium was assayed by ELISA and western. Western analysis of the secreted GIPF showed that no degradation of the protein had occurred. Western analysis was performed using a purified anti-GIPF polyclonal antibody, and the detection of the protein by ELISA was performed using a purified chicken anti-GIPF polyclonal antibody as the capture antibody, and the rabbit anti-GIPF polyclonal antibody as the detection antibody. The rabbit and chicken polyclonal antibodies were raised against the whole protein. Growth of GIPFwt in the bioreactors yielded 2.6-3 mg/l.

Ultrafiltration-Diafiltration of the medium containing the secreted GIPFwt protein was harvested by centrifugation. Protease inhibitors 1 mM EDTA and 0.2 mM Pefabloc (Roche, Basel, Switzerland) were added to prevent degradation of GIPF. The medium was filtered through a 0.22 μm PES filter (Corning), and concentrated 10-fold using TFF system (Pall Filtron) or hollow-fiber system (Spectrum) with 10 kDa cut-off membrane. The buffer of the concentrated medium was exchanged with 20 mM sodium phosphate, 0.3 M Arg, pH 7. The addition of 0.3 M Arg in the phosphate buffer is crucial to keep GIPFwt fully soluble at pH 7 during purification. After ultrafiltration and diafiltration, a mammalian protease inhibitor cocktail (Sigma) was added at 1:500 (v/v) dilution.

Q anion exchange chromatography: an anion exchange Q Sepharose HP column (Amersham) was equilibrated with 20 mM sodium phosphate (NaP) buffer at pH7.0 and containing 0.3 M Arg. The 10-fold concentrated and buffer-exchanged medium was filtered with 0.22 μm PES filter and loaded onto the Q Sepharose column to bind impurities and nucleic acids.

SP cation exchange chromatography: the Q-Sepharose flow through containing GIPFwt was collected and loaded onto a cation exchange SP Sepharose HP (Amersham), which bound the GIPF protein. The SP Sepharose column was washed with 15 column volumes (CV) of 20 mM NaP, 0.3 M Arg, 0.1M NaCl, pH 7, and GIPF was eluted with a gradient of 0.1 M to 0.7 M NaCl over 40 column volumes. The fractions were analyzed by SDS-PAGE and Western blot. Fractions containing GIPFwt were analyzed and pooled. The buffer of the pooled fractions was exchanged with 20 mM sodium phosphate, pH 7, 0.15 M NaCl. The purity of the purified protein was determined to be 92-95% when analyzed by Comassie staining of an SDS-gel. The protein was concentrated to 1 mg/ml, and passed through a sterile 0.22 μm filter and stored at −80° C.

The yield obtained at the end of each step in the purification process was quantified by ELISA and by the Bradford assay, and the percent recovery of GIPF protein was calculated as shown in Table 2.

TABLE 2

| Steps | Step Recovery | Overall Recovery |
|---|---|---|
| Media | 100% | 100% |
| Concentration/Diafiltration | | |
| Q Anion Exchange | 95% | 95% |
| SP Cation Exchange | 75% | 71% |
| Final Formulation and filter | 98% | 70% |
| | | 48% (dominant |
| | | mature form only) |

The endotoxin level of the final formulated GIPF protein solution was analyzed using chromogenic LAL (Limulus Amebocyte Lysate) assay kit (Charles River), and determined to be 0.24 EU per mg of GIPF.

### C. Characteristics of Purified Recombinant GIPF

SDS-PAGE analysis of the purified GIPF protein (GIPFwt) was performed under reducing and non-reducing conditions, and showed that the V5-His tagged GIPF proteins derived from 293 cells exists as a monomer. GIPFwt protein is glycosylated and migrate on SDS-PAGE under non-reducing conditions with a molecular weight (MW) of approximately 38 kDa. Matrix-assisted laser desorption/ionization mass spectroscopy (MALDI) showed that the respective molecular weight for GIPFwt is 32.9 kDa, while the theoretical molecular weight for GIPF wt that lack the signal peptide is 26.8 kDa. The discrepancy in the molecular weights suggested that it might have been accounted for by the glycosylation of the protein. Subsequently, complete deglycosylation of N-linked and O-linked oligosaccharides was performed using N- and O-glycanase (Prozyme, San Leandro, Calif., USA) according to the manufacturer's instructions. SDS-PAGE analysis of the deglycosylated protein resulted in a decrease in apparent molecular weight of 4-5 kDa.

N-terminal sequence analysis showed that HEK293 cells produced two forms of GIPFwt polypeptide: the dominant mature form (SEQ ID NO: 6) which corresponds to the GIPF protein of SEQ ID NO: 4 that lacks the signal sequence, and the mature form (SEQ ID NO: 7), which corresponds to the GIPF protein of SEQ ID NO: 3 that lacks both the signal peptide and the furin cleavage sequence. The two forms separated well on the SP column, and were expressed at a ratio of mature to dominant mature forms of approximately 1:2. The dominant mature form was used to test the effect of GIPF in the animal models and in vitro tests.

In summary, the purification processes yield a GIPFwt that is 92-95% pure. The overall recovery of the dominant mature form of GIPF is approximately 50%. Addition of 0.5 M NaCl to the buffer during the purification process of media diafiltration and Ni column is crucial to keep GIPF fully soluble at pH 7. For binding GIPF onto the SP column, NaCl was removed, and 0.3 M Arg was added to maintain high solubility and increase protein recovery.

The dominant mature and mature form of GIFP wt were used to test the biological activity of GIPF in vivo and in vitro.

### Example 3

### The Pharmacokinetics (PK) of Recombinant GIPF Protein Expressed in HEK293 and CHO Cells

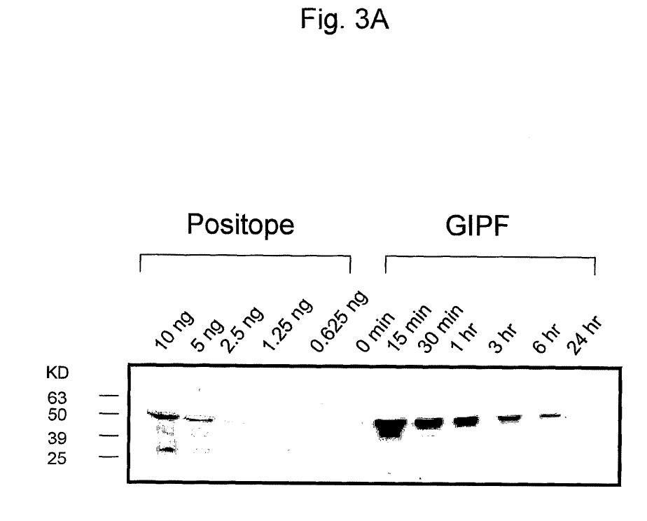

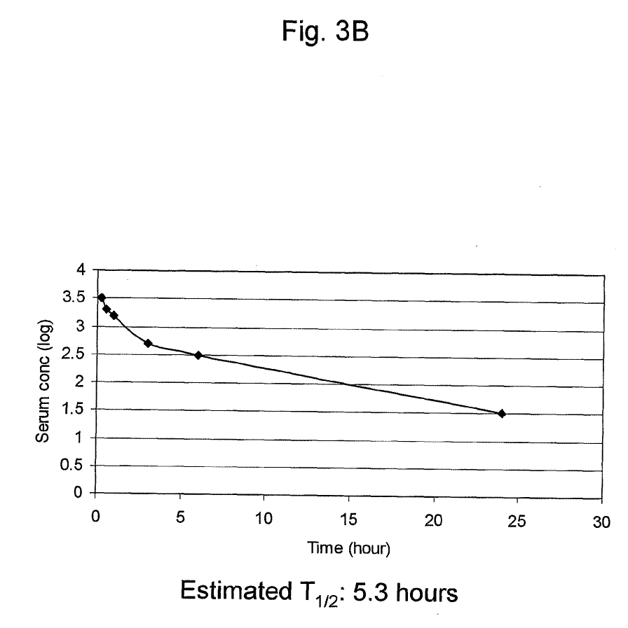

The pharmacokinetics (PK) of recombinant GIPF V5His6-tagged protein (GIPFt) were determined in mice. 6-8 weeks old BALB/c mice were injected i.v. via the tail vein with single dose of either 40 mg/KG GIPFt protein or formulation buffer as control. Blood was withdrawn at 0, 30 min, 1 hr, 3 hr, 6 hr and 24 hr after injection and serum protein level at each time point was analyzed by Western analysis using anti V5 antibody (Invitrogene Inc., Carlsbad, Calif.) FIG. 3 A shows that no significant degradation of serum GIPF protein was detected. The half-life of GIPF protein in serum was calculated by semi logarithmic plot of the protein concentration after injection using Positope (Invitrogene Inc., Carlsbad, Calif.) as a standard V5 tagged protein, and was estimated to be 5.3 hours (FIG. 3 B).

### Example 4

### Anti-Tumor Effect of GIPF in NIH3T3 Transfectants Transferred Mice

A. Preparation of pcmvGIPF-IRES-GFP

Following the digestion of pIRES2-EGFP (BD Bioscience Clontech) with EcoRI and NotI, the fragment including the IRES-GFP region was purified by 0.8% agarose gel electrophoresis and QIA quick Gel Extraction Kit (QIAGEN). The purified fragment (IRES-GFP) was ligated to pcDNA3 (Invitrogen) that was digested with EcoRI and NotI, and treated with calf intestine alkaline phosphatase to dephosphorylate its both ends. The ligation mixture was transfected to DHα and the DNA samples prepared from the resultant transformants were analyzed by nucleotide sequencing to confirm the structure of inserted fragment. The clone including a fragment with a correct nucleotide sequence was selected (pIRES-GFP).

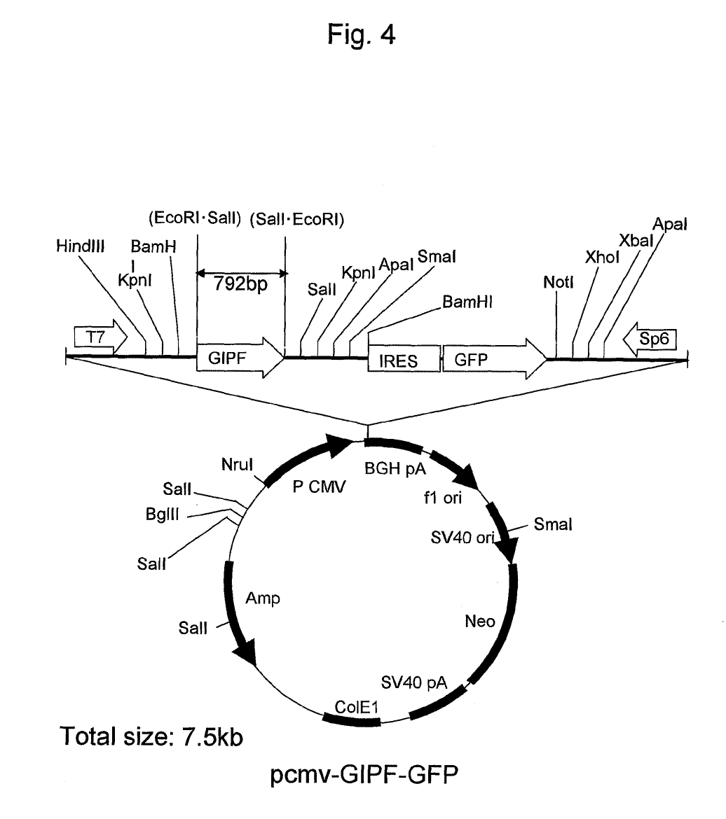

The GIPF fragment (0.81 kb, SalI-SalI) was prepared by using a following primer pair and a full-length GIPF cDNA derived from human fetal skin cDNA library (Invitrogen) as a template: GIPF-F, ACGCGTCGACCCACATGCGGCTTGGGCTGTGTGT (including SalI site and Kozak sequence at the 5′ end; SEQ ID NO: 10) and GIPF-R, ACGCGTCGACGTCGACCTAGGCAGGCCCTG (including SalI site at the 5′ end; SEQ ID NO:11). Subsequently, the 0.81 kb GIPF fragment was digested with SalI and treated with Blunting high (TOYOBO) for blunting its both ends. The resultant DNA fragment including GIPF coding region was purified by 0.8% agarose gel electrophoresis. This GIPF fragment was ligated to the pIRES-GFP vector that was subjected to digestion with EcoRI, treatment with Klenow fragment (TAKARA BIO) for blunting its both ends, and further treatment with E. Coli C75 alkaline phosphatase to dephosphorylate its both ends. The ligation mixture was transfected to DHα and the DNA samples prepared from the resultant transformants were analyzed by nucleotide sequencing to confirm the structure of inserted fragment. The clone including the GIPF fragment in same orientation to CMV promoter was selected (pcmvGIPF-IRES-GFP: FIG. 4).

B. Preparation of pcmvEPO-IRES-GFP

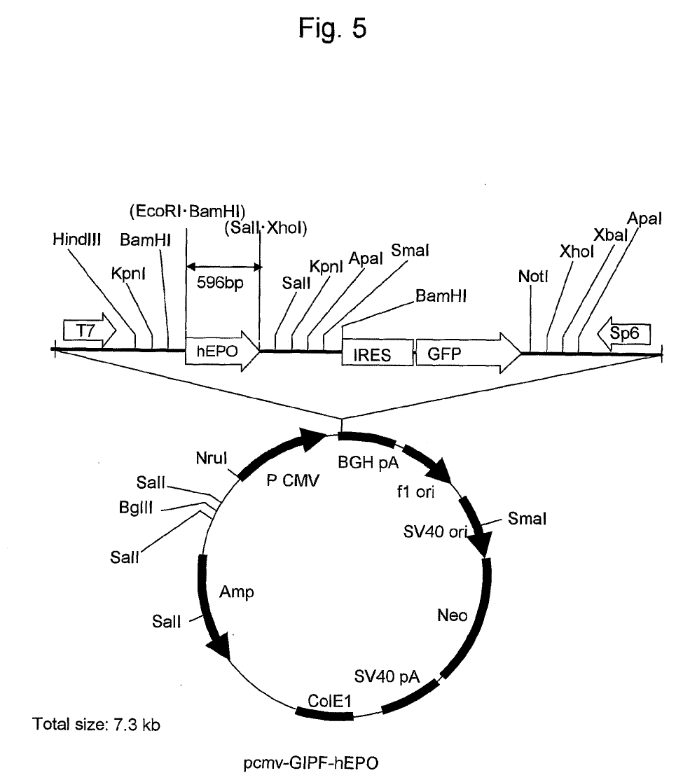

Following the digestion of pLN1/hEPO (Kakeda et al., Gene Ther., 12: 852-856, 2005) with BamHI and XhoI, the fragment including the human erythropoietin (hEPO) coding region was treated with Blunting high (TOYOBO) for blunting its both ends. Subsequently, the 0.6 kb HEPO fragment was purified by 0.8% agarose gel electrophoresis and QIA quick Gel Extraction Kit (QIAGEN). This hEPO fragment was ligated to the pIRES-GFP vector that was subjected to digestion with EcoRI, treatment with Klenow fragment (TAKARA BIO) for blunting its both ends, and further treatment with E. Coli C75 alkaline phosphatase to dephosphorylate its both ends. The ligation mixture was transfected to DHα and the DNA samples prepared from the resultant transformsants were analyzed by nucleotide sequencing to confirm the structure of inserted fragment. The clone including the HEPO fragment in same orientation to CMV promoter was selected (pcmvEPO-IRES-GFP: FIG. 5).

C. Preparation of pcmvGIPF-IRES-GFP and pcmvEPO-IRES-GFP Plasmid DNA for Electroporation to NIH3T3

The plasmid DNA of pcmvGIPF-IRES-GFP and pcmvEPO-IRES-GFP was digested with BglII in the reaction mixture containing 1 mM spermidine (pH7.0, Sigma) for 5 hours at 37° C. The reaction mixture was then subjected to phenol/chloroform extraction and ethanol precipitation (0.3M NaHCO₃) for 16 hours at −20° C. The linearized vector fragment was dissolved in Dulbecco's phosphate-buffered saline (PBS) buffer and used for the following electroporation experiments.

The linearized pcmvGIPF-IRES-GFP and pcmvEPO-IRES-GFP vector were transfected into NIH3T3 cells (obtained from Riken Cell Bank, RCB0150). The NIH3T3 cells were treated with trypsin and suspended in PBS at a concentration of 5×10⁶ cells/ml, followed by electroporation using a Gene Pulser (Bio-Rad Laboratories, Inc.) in the presence of 10 μg of vector DNA. A voltage of 350V was applied at a capacitance of 500 μF with an Electroporation Cell of 4 mm in length (165-2088, Bio-Rad Laboratories, Inc.) at room temperature. An electroporated cells were inoculated into Dulbecco-modified Eagle's MEM (DMEM) supplemented with 10% of fetal bovine serum (FBS) in a tissue culture plastic plate of 100 mm². After one day the medium was replaced with a DMEM supplemented with 10% FBS and containing 800 μg/ml of G418 (GENETICIN, Sigma). Over 200 of G418-resistant colonies were formed in each 100 mm² plate after two weeks. The resultant colonies were treated with trypsin, mixed for each 100 mm² plate and inoculated again into a plate of 100 mm² and cultured for propagation. Two pools (A-2, A-5) for mixed transfectants by pcmvGIPF-IRES-GFP vector and two pools (C-3, D-3) for mixed transfectants by pcmvEPO-IRES-GFP vector were used for the following experiments. 6×10⁵ cells of mixed transfectants for each pool were suspended in PBS supplemented with 5% fetal bovine serum (FBS; Gibco) and 1 μg/ml of propidium iodide (Sigma, St. Louis, Mo.), and analyzed by FACSVantage (Becton Dickinson, Franklin Lakes, N.J.). The GFP-positive cells exhibiting high fluorescence intensity (upper 15%) was sorted and cultured for further propagation of pooled transfectants with high-level expression of GFP (A-2GH, A-5GH, C-3GH, D-3GH). Two (A-2GH, A-5GH) and one (D-3GH) of pooled transfectant with high-level expression of GFP and non-transfectant NIH3T3 cell were used for the following transplantation experiments.

### D. Determination of Growth Rate of Pooled Transfectants

To determine the growth rate in culture 1×10⁵ cells of each of four pooled transfectant with high level expression of GFP or control NIH3T3 was inoculated into DMEM supplemented with 10% FBS in a tissue culture plastic plate of 100 mm². When the culture reached sub-confluence the cells were treated with trypsin, and one tenth (Exp. 1: 10×) or one twentieth (Exp. 2: 20×) of total cells were re-inoculated into DMEM supplemented with 10% FBS in a tissue culture plastic plate of 100 mm². Repeated culture and inoculation were performed for 532 hr for Exp. 1 and 561 hr for Exp.2 according to the above procedure. Subsequently, total cell number in 100 mm² dish was counted for each experiment and the doubling time for each pooled transfectants with high level expression of GFP or control NIH3T3 was calculated in each experiment. The results are presented in Table 3. In conclusion, there is no change in in vitro growth rate of pooled transfectants with high-level GFP expression when compared to that of control NIH3T3.

TABLE 3
Doubling time of each pooled transfectants

| | |
|---|---|
| Pool | Doubling Time (hr) |

Exp. 1

| | |
|---|---|
| wild-type NIH3T3 | 17.6 |
| A-2GH | 18 |
| A-5GH | 17.9 |
| C-3GH | 17.8 |
| D-3GH | 17.8 |

Exp. 2

| | |
|---|---|
| wild-type NIH3T3 | 19 |
| A-2GH | 19.1 |
| A-5GH | 19 |
| C-3GH | 18.8 |
| D-3GH | 19.2 |

### E. Anti-Tumor Effect of GIPF in NIH3T3 Transfectants Transferred Mice

The effect of the GIPF expressing NIH3T3 cell was examined using a cell transfer mouse model according to the following method.

4 to 6 scid mice (purchased from CLEA Japan) were grouped into 5 groups as follows, 1) SCa group: A-2GH GIPF expressing NIH3T3 cell transferred group, 2) SCb group: A-5GH GIPF expressing NIH3T3 cell transferred group, 3) SCc group: D-3GH human erythropoietin (HEPO) expressing NIH3T3 cell transferred group, 4) SCd group: wild-type NIH3T3 cell transferred group and 5) SCe group: DMEM injected group as control. GIPF and HEPO expressing cells or wild-type NIH3T3 cells were intravenously (iv) and intraperitoneally (ip) transferred at 5×10⁶ cells/mouse in 300 to 600 μl of DMEM to scid mice at 5-week-old. In the SCe group, 300 to 600 μl of DMEM was also iv or ip injected. Mortality and clinical observations for general health and appearance were carried out once daily. Mice that showed moribund condition and were sacrificed for pathological analysis, serum chemical analysis and histopathology. All survived animals were weighed once in every week after cell transfer. For hematological analysis, blood samples from all mice were taken at 5-week-old prior to cell transfer and blood samples from all survived mice were taken every 2 weeks after cell transfer. Measurements of hematology parameters were carried out using collected blood samples by Advia 120 apparatus (Bayel-Medical). For pathological analysis, all survived mice were sacrificed at 42 days after cell transfer. Mice were anesthetized with diethyl ether and blood samples were taken from inferior vena cava. For collection of serum samples, blood samples were transferred to Microtainer (Becton Dickinson) and stored at room temperature for 30 minutes then centrifuged 8,000 rpm for 10 minutes. The serum biochemistry parameters were examined with collected serum samples. At necropsy, external appearance, abdominal cavities, subcutaneous tissues, thoracic cavities and organs including gastrointestinal tissues were examined. Organ weights were measured and organs and tissues including sarcomas or tumors were macroscopically examined and fixed in 10% neutral buffered formalin for histopathologic analysis. Hematoxyline-Eosin stained specimens were prepared from spleen, liver, heart, pancreas, gastrointestinal tract, lymph node or other tissues that obvious abnormality were observed. Daily observation revealed formation of small knobs or tumors in subcutaneous layer or under muscular layer in intraperitoneally transferred mice from 9 to 10 weeks after cell transfer. It was expected that these knobs or tumors were aggregated NIH3T3 cell mass which was transplanted on peritoneum. Such tumorigenesis was observed in all groups but mice in SCc (SCc2) and SCd (SCd2) groups developed larger tumors in earlier period compared to mice in SCa or SCb groups (Table 4).

TABLE 4
Tumor formation or growth detected
in each groups after cell transfer

| | ID | | days after | |
|---|---|---|---|---|
| Group | number | route | cell transfer | part of tumor formation/size |
| Sca | SCa3 | ip | 34 | left hind leg/9 × 6 × 4 mm |
| | | | 38 | left hind leg/9 × 9 × 5 mm |
| | | | 41 | left hind leg/12 × 12 × 7 mm |
| Scb | SCb4 | ip | 38 | hypogastrium/4 × 5 × 2 mm |
| | | | 41 | hypogastrium/4 × 4 × 3 mm |
| | SCb5 | ip | 41 | hypogastrium/5 × 4 × 4 mm |
| | SCb6 | ip | 41 | hypogastrium/5 × 5 × 4 mm |
| SCc | SCc2 | iv | 33 | left hind leg/20 × 15 × 10 mm |
| | SCc3 | ip | 33 | hypogastrium/10 × 6 × 4 mm |
| | SCc4 | ip | 34 | left hind leg/3 × 4 × 1 mm |
| SCd | SCd2 | iv | 33 | left buttock/17 × 12 × 10 mm |
| | SCd4 | ip | 34 | left hind leg/8 × 5 × 4 mm |
| | | | 38 | left hind leg/9 × 5 × 3 mm |

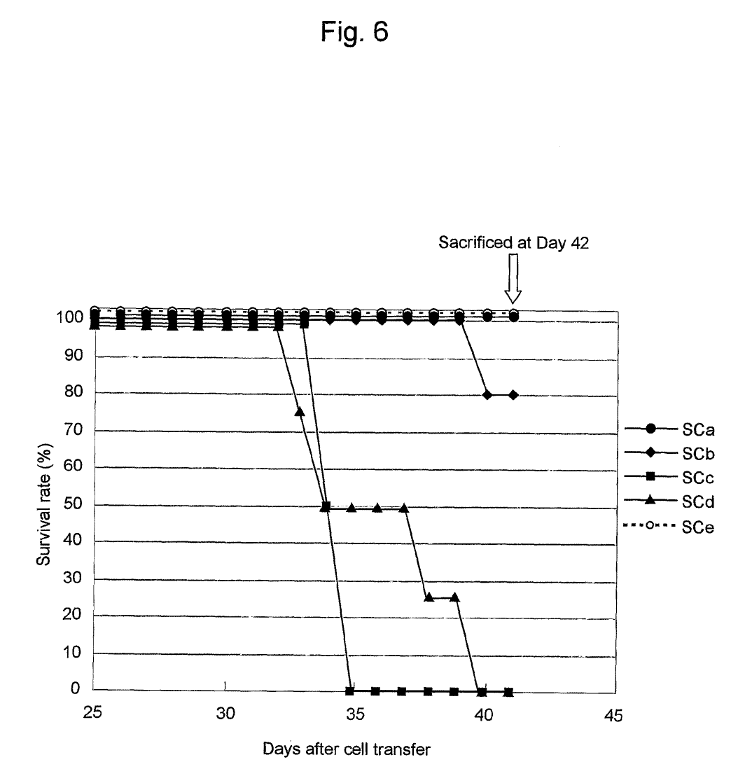

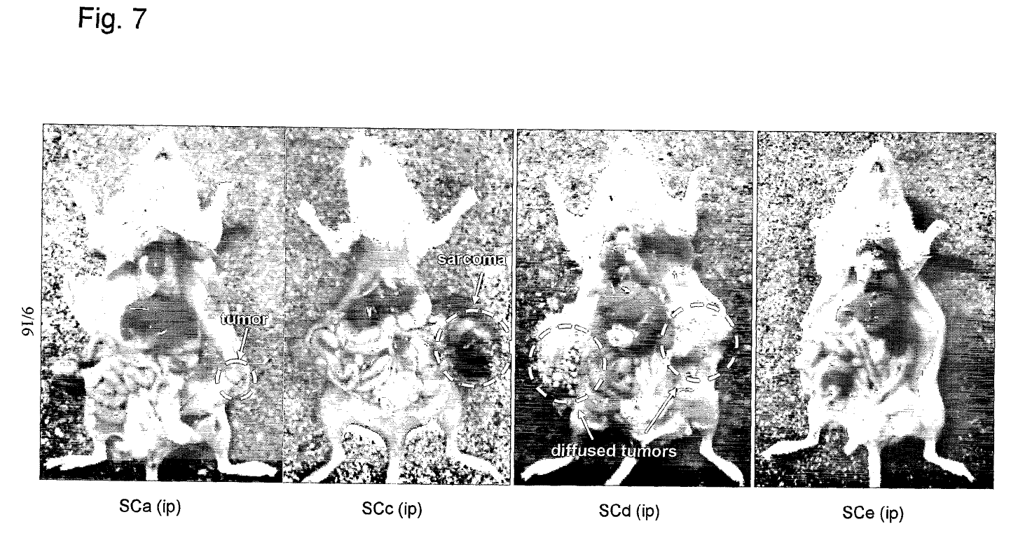

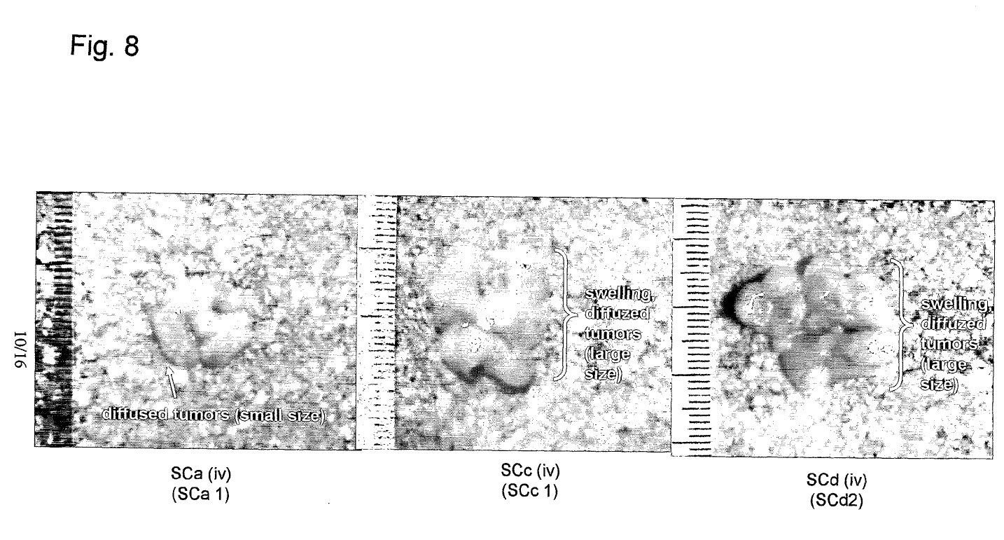

In the general clinical observation, some mice displayed coarse hair, abnormal respiration, hypothermia or anemia. It was expected that this general health deterioration resulted in transferred cell tumorigenesis in vivo. Body weight curve of each groups did not show obvious difference between the groups except in SCc group that showed rapid decrease of body weight at 5 weeks after cell transfer (data not shown). FIG. 6 shows the results of survival curve of cell transferred mice in each group. In the SCc group, survival rate was rapidly reduced at 34 days after cell transfer (survival rate 50%) and all mice were dead at 35 days after cell transfer. In the SCd group, survival rate was gradually reduced from 33 days after cell transfer and all mice were dead at 40 days after cell transfer. In FIG. 6, when GIPF expressing NIH3T3 cells were transferred, survival rates were relatively higher than SCc or SCd groups over 40 days after cell transfer. Only slight reduce of survival rate was observed in SCb group compared to SCe control group, furthermore all mice were survived in SCa group (SCa 100% and SCb 80% mice were survived at 40 days after cell transfer). In hematological analysis, increase of red blood cell count (RBC) was observed in SCc group from 2 weeks after cell transfer. At 4 weeks after cell transfer, average RBC was 13.32×10⁶ cells/uL and 10.47×10⁶ cells/uL in SCc group and SCd group respectively. This means that human erythropoietin transgene was expressed in transferred NIH3T3 cells in vivo and as expected transgene expression had effect on recipient mice physiology in this model. As shown in Table 4, ip transferred mice developed small tumor masses that were scattered in their abdominal cavity furthermore sarcoma and hematoma were observed in peritoneum, mesenterium and adipose tissue. But ip cell transferred mice in SCa group, the large sarcoma and hematoma were not observed compared to other groups (Table 5 and FIG. 7). On the other hand tumors were developed in the lungs of iv cell transferred mice in each group (Table 5 and FIG. 8). But the size of tumor was smaller in SCa and SCb groups compared to SCc or SCd groups (FIG. 8).

These data suggests that GIPF expression suppresst the growth of NIH3T3 tumor growth in vivo. Transferred cells were distributed in the abdominal cavity in ip or lung in iv cell transferred mice and developed tumors or sarcomas. The difference among cell types is affected the tumor growth after distribution. Human EPO or wild-type NIH3T3 cells have no cell-death-inducing or anti-tumor activity against transferred cell tumor development. On the other hand, reduce of tumor number and size observed in GIPF expressing cell transferred mice compared to the HEPO expressing and wild-typ NIH3T3 cell transferred mice. It is expected that GIPF has some cell-death-inducing, anti-tumor or anti-angiogenesis activity. GIPF was produced in transferred NIH3T3 cells and it affected in autocrine or paracrine manner to suppress tumor growth or development in this model. Therefore, mortality of GIPF expressing cell received mice was reduced because of GIPF anti-tumor development activity.

TABLE 5
Tumors observed in NIH3T3 cell transferred mice at necropsy

| | | | tumor developed regions | | | |
|---|---|---|---|---|---|---|
| | ID | | | abdominal | | |
| group | number | route | subcutaneous | cavity | peritoneum | lung |
| SCa | SCa 1 | iv | ND | ND | ND | diffused tumors |
| | | | | | | (slight) |
| SCa | SCa 2 | iv | ND | ND | ND | diffused tumors |
| SCa | SCa 3 | ip | tumorigenesis | diffused | ND | ND |
| | | | | tumors | | |
| SCa | SCa 4 | ip | ND | ND | ND | ND |
| SCa | SCa 5 | ip | ND | diffused | ND | ND |
| | | | | tumors | | |
| SCa | SCa 6 | ip | ND | diffused | ND | ND |
| | | | | tumors | | |
| SCb | SCb 1 | iv | ND* | ND* | ND* | ND* |
| SCb | SCb 2 | iv | ND | ND | ND | ND |
| SCb | SCb 3 | ip | ND | diffused | ND | ND |
| | | | | tumors | | |
| SCb | SCb 4 | ip | tumorigenesis | diffused | tumorigenesis | diffused tumors |
| | | | | tumors | | |
| SCb | SCb 5 | ip | tumorigenesis | diffused | tumorigenesis | ND |
| | | | | tumors | | |
| SCb | SCb 6 | ip | tumorigenesis | diffused | ND | ND |
| | | | | tumors | | |
| SCc | SCc 1 | iv | ND | ND | ND | swelling, diffused |
| | | | | | | tumors |
| SCc | SCc 2 | iv | tumorigenesis, | ND | ND | swelling, diffused |
| | | | sarcoma, hemetoma | | | tumors |
| SCc | SCc 3 | ip | tumorigenesis | diffused | tumorigenesis | swelling, diffused |
| | | | | tumors | | tumors |
| SCc | SCc 4 | ip | tumorigenesis | diffused | ND | ND |
| | | | | tumors | | |
| SCd | SCd 1 | iv | ND* | ND* | ND* | diffused tumors |
| SCd | SCd 2 | iv | tumorigenesis | ND | ND | swelling (severe), |
| | | | | | | diffused tumors |
| SCd | SCd 3 | ip | ND | diffused | tumorigenesis | ND |
| | | | | tumors | | |
| SCd | SCd 4 | ip | tumorigenesis | diffused | tumorigenesis | ND |
| | | | | tumors | | |
| SCe | SCe 1 | iv | ND | ND | ND | ND |
| SCe | SCe 2 | iv | ND | ND | ND | ND |
| SCe | SCe 3 | ip | ND | ND | ND | ND |
| SCe | SCe 4 | ip | ND | ND | ND | ND |

ND: not detected
*autopsy was performed after death

### Example 5

### Effect of GIPF on Tumor-Bearing Mice

The effect of GIPF on tumor growth was examined using a tumor-bearing mouse model according to the following method.

To prepare the donor tumor blocks, Sw620 (Human lympho node metastasis from colorectal adenocarcinoma; epitherial) cells were subcutaneously transplanted in the dorsal areas at 5×10⁶/mouse to 7-week-old Balb/c nude mice (purchased from CLEA Japan). When the tumor volume became about 400 mm³, tumors were cut and trimmed to about 2×2×2 mm size with crossed scalpels. Tumor block of Sw620 were subcutaneously transplanted in the dorsal areas to 9-week-old Balb/c nude mice (purchased from CLEA Japan). When the tumor volume became about 100 mm³ or 200 mm³, the mice were grouped so that the groups each consisted of six mice and had an even average tumor volume.

COLO205 (Human ascites from metastatic colorectal adenocarcinoma; epitherial) and HT29 (Human colorectal adenocarcinoma; epitherial) cells were subcutaneously transplanted in the dorsal areas at 2×10⁶/mouse to 10-week-old Balb/c nude mice (purchased from CLEA Japan). When the tumor volume became about 50 mm³ or 150 mm³, the mice were grouped so that the groups each consisted of six mice and had an even average tumor volume.

GIPF was injected intravenously at 100 μg/mouse (dissolved in 100 μl of PBS), daily for 7 days after grouping. The same volume of PBS was used as a negative control.

Tumor dimensions and body weights were measured 3× per week and tumor volume is calculated as width×width×length×0.52.

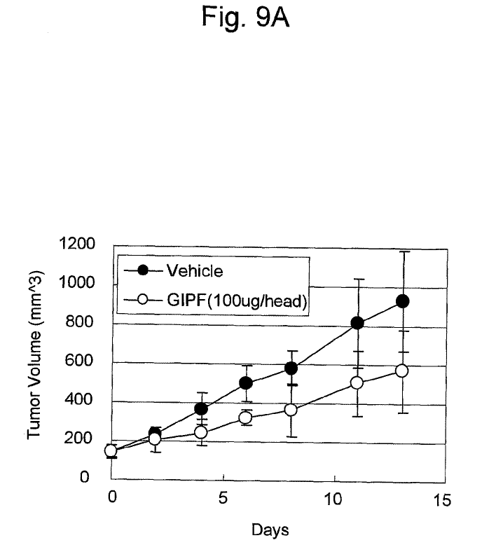

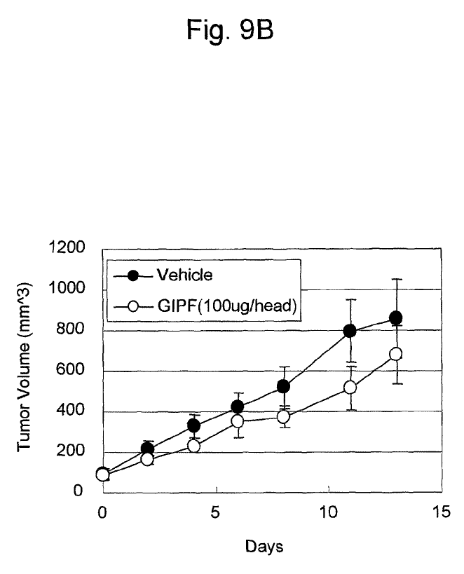

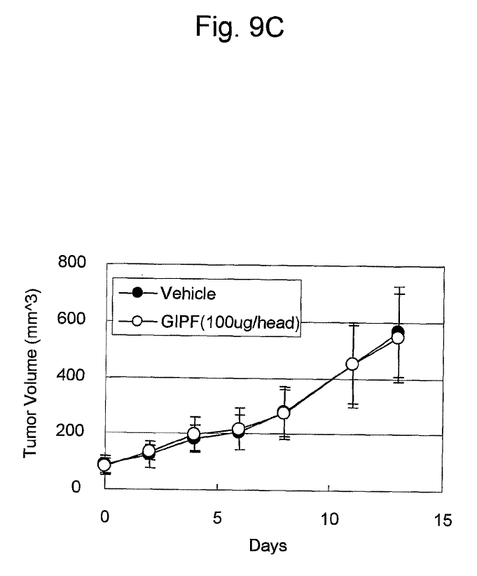

FIG. 9 shows the results of the above experiments. The administration of GIPF did not only enhance the growth of the all three tumors, but also, significantly induced anti-tumor effects in the Sw620 and COLO205.

FIG. 9 A shows the results of measuring the Sw620 tumor size when GIPF were administered at 100 μg/mouse daily for 7 days.

FIG. 9 B shows the results of measuring the COL0205 tumor size when GIPF were administered at 100 μg/mouse daily for 7 days.

FIG. 9 C shows the results of measuring the HT29 tumor size when GIPF were administered at 100 μg/mouse daily for 7 days.

### Example 6

### Effect of GIPF on the Proliferation of Normal Human Endothelial Cells

To investigate the proliferative effect on in vitro, the effect of recombinant GIPF was tested on the proliferation of normal human endothelial cells. Primary human umbilical vein endothelial cells (HUVECS) and human dermal microvascular endothelial cells (HMVECs) were purchased from Cambrex (Walkersville, Md.) and grown in Cambrex' endothelial cell growth media. The rate of cell proliferation of the HUVECs and HMVECs was measured by assaying the incorporation of 3H-thymidine.

Briefly, HUVECs or HMVECs were seeded in collagen-coated 96-well plates at 4,000 cells per 200 μL/well in endothelial basal medium-2 (EBM2; Cambrex (Walkersville, Md.) containing 5% FBS. After 24 hours, GIPF (3-1000 ng/ml) was added followed by 20 ng/mL VEGF and the cells were cultured for 78 hours. 3H-thymidine (1 μCi/mL) was added and the cells were cultured for a further 14 hours. They were then harvested and their radioactivity was measured using a liquid scintillation counter (Wallac 1205 Beta Plate; Perkin-Elmer Life Sciences, Boston, Mass.). The rate of proliferation of the GIPF-treated cells was compared to that of untreated cells.

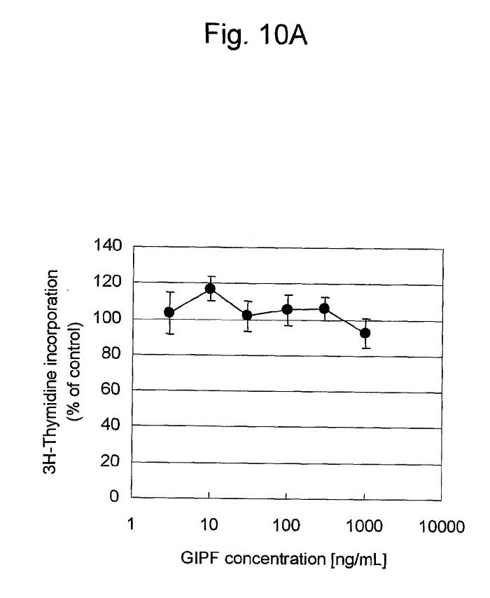

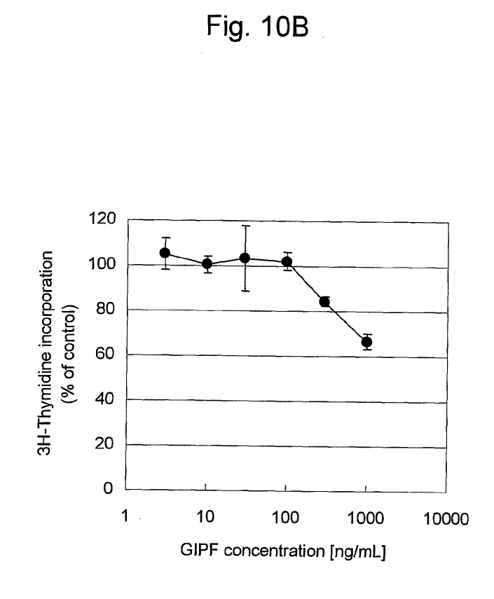

FIG. 10 shows the results of the above experiments. GIPF inhibited VEGF-driven HMVEC proliferation, but not HUVEC proliferation.

FIG. 10, GIPF inhibited VEGF-driven HMVEC proliferation but not HUVEC proliferation. HUVECs or HMVECs were seeded and cultured for 24 hours. Cells were incubated with GIPF before stimulation with 20 ng/mL VEGF (•). The cells were then cultured for 78 hours followed by incubation with 3H-thymidine (1 μCi/mL) for 14 hours. The incorporated radioactivity of the cells was measured using a liquid scintillation counter. Points, means (n=3); bars, SD.

### Example 7

### Effect of GIPF on the Migration of Normal Human Endothelial Cells

To investigate the effect of GIPF on migration of normal human endothelial cells, migration of HMVECs was measured by matrigel invasion chamber (BD Biosciences) systems. Primary human dermal microvascular endothelial cells (HMVECs) were purchased from Cambrex (Walkersville, Md.) and grown in Cambrex' endothelial cell growth media.

Cell migration was assayed in 24-well Matrigel invasion chambers. The Matrigel Invasion Chambers consist of BD falcon™ cell culture inserts containing an 8 micron pore size PET membrane that has been treated with Matrigel Matrix. Briefly, HMVECs were harvested and pretreated with GIPF (10 or 100 ng/ml) in control medium (EBM2 containing 0.1% BSA) for 30 min in suspension. 2×10⁵ cells were loaded to the top of each invasion chamber and were allowed to migrate to the underside of the chamber for 4 h at 37° C. in the presence or absence of VEGF (5 or 50 ng/ml) in the lower chamber. Cells were fixed and stained with Diff-Quick (Sysmex corp.) Non-migrated cells on the top of the filters were wiped off and migrated cells attached to the bottom of the filter were counted using bright-field microscopy. Each determination represents the average of two individual wells. Migration was normalized to percent migration, with migration to VEGF representing 100% migration.

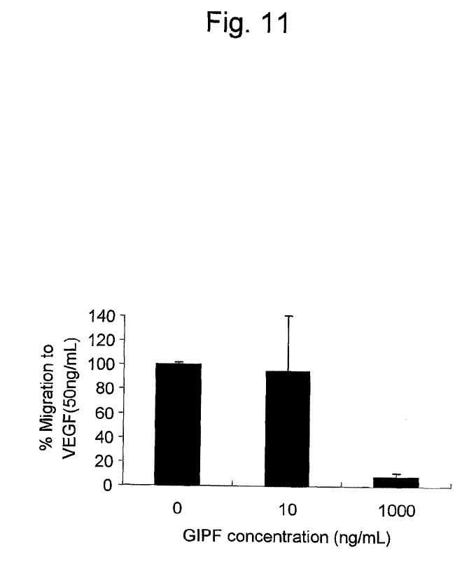

FIG. 11 shows the results of the above experiments. GIPF inhibited VEGF-induced HMVEC migration.

FIG. 11, GIPF inhibited VEGF-induced HMVEC migration. Cell migration is expressed as percentage of the maximal migration induced by VEGF. Dashed line indicates basal migration levels, in the absence of VEGF. Error bars indicate SDs. **, P<0.01 compared with VEGF alone as determined using t test for unpaired data.

This specification hereby incorporates all the publications, patents and patent applications cited in this specification in their entirety by reference.

1-9. (canceled)

10. A method of treating a tumor, which comprises administering to a human, as an active ingredient, a molecule that is (a) a human R-spondin or (b) a fragment thereof that has human R-spondin activity, wherein said molecule is selected from (i) to (xii) below:
(i) R-spondin 1 having the amino acid sequence represented by SEQ ID NO:3 or a fragment thereof which has human R-sopndin 1 activity;
(ii) R-spondin 1 variant having the amino acid sequence derived from the amino acid sequence represented by SEQ ID NO:3 by deletion, substitution, or addition of 1 to 5 amino acids and has R-spondin 1 activity, or a fragment thereof which has human R-spondin 1 activity;
(iii) R-spondin 1 having the amino acid sequence represented by SEQ ID NO:6 or a fragment thereof which has human R-sopndin 1 activity;
(iv) R-spondin 1 variant having the amino acid sequence derived from the amino acid sequence represented by SEQ ID NO: 6 by deletion, substitution, or addition of 1 to 5 amino acids and has R-spondin 1 activity, or a fragment thereof which has human R-spondin 1 activity;
(v) R-spondin 1 having the amino acid sequence represented by SEQ ID NO: 7 or a fragment thereof which has human R-sopndin 1 activity;
(vi) R-spondin 1 variant having the amino acid sequence derived from the amino acid sequence represented by SEQ ID NO:7 by deletion, substitution, or addition of 1 to 5 amino acids and has R-spondin 1 activity, or a fragment thereof which has human R-spondin 1 activity;
(vii) R-spondin 2 having the amino acid sequence represented by 22nd amino acid to 206th amino acid of SEQ ID NO:13 or a fragment thereof which has human R-sopndin 2 activity;
(viii) R-spondin 2 variant having the amino acid sequence derived from the amino acid sequence represented by 22nd amino acid to 206th amino acid of SEQ ID NO:13 by deletion, substitution, or addition of 1 to 5 amino acids and has R-spondin 2 activity, or a fragment thereof which has human R-sopndin 2 activity;
(ix) R-spondin 3 having the amino acid sequence represented by 22nd amino acid to 272nd amino acid of SEQ ID NO: 15 or a fragment thereof which has human R-sopndin 3 activity;
(x) R-spondin 3 variant having the amino acid sequence derived from the amino acid sequence represented by 22nd amino acid to 272nd amino acid of SEQ ID NO:15 by deletion, substitution, or addition of 1 to 5 amino acids and has R-spondin 3 activity, or a fragment thereof which has human R-sopndin 3 activity;
(xi) R-spondin 4 having the amino acid sequence represented by SEQ ID NO:17 or a fragment thereof which has human R-spondin 4 activity; and
(xii) R-spondin 4 variant having the amino acid sequence derived from the amino acid sequence represented by SEQ ID NO:17 by deletion, substitution, or addition of 1 to 5 amino acids and has R-spondin 4 activity, or a fragment thereof which has human R-spondin 4 activity.

11. A method of treating tumor, which comprises administering to a human, as an active ingredient, a DNA that encodes (a) human R-spondin or (b) a fragment thereof that has human R-spondin activity, wherein said DNA is selected from (i) to (xii) below:
(i) a DNA encoding R-spondin 1 having the nucleotide sequence encoding the amino acid sequence represented by SEQ ID NO:3 or a fragment thereof which has human R-sopndin 1 activity;
ii) a DNA of R-spondin 1 variant having the nucleotide sequence encoding the amino acid sequence derived from the amino acid sequence represented by SEQ ID NO:3 by deletion, substitution, or addition of 1 to 5 amino acids and has R-spondin 1 activity, or a fragment thereof which has human R-spondin 1 activity;
iii) a DNA of R-spondin 1 having the nucleotide sequence encoding the amino acid sequence represented by SEQ ID NO:6 or a fragment thereof which has human R-sopndin 1 activity;
iv) a DNA of R-spondin 1 variant having the nucleotide sequence encoding the amino acid sequence derived from the amino acid sequence represented by SEQ ID NO: 6 by deletion, substitution, or addition of 1 to 5 amino acids and has R-spondin 1 activity, or a fragment thereof which has human R-spondin 1 activity;
v) a DNA of R-spondin 1 having the nucleotide sequence encoding the amino acid sequence represented by SEQ ID NO: 7 or a fragment thereof which has human R-sopndin 1 activity;
vi) a DNA of R-spondin 1 variant having the nucleotide sequence encoding the amino acid sequence derived from the amino acid sequence represented by SEQ ID NO:7 by deletion, substitution, or addition of 1 to 5 amino acids and has R-spondin 1 activity, or a fragment thereof which has human R-spondin 1 activity;
vii) a DNA of R-spondin 2 having the nucleotide sequence encoding the amino acid sequence represented by 22nd amino acid to 206th amino acid of SEQ ID NO:13 or a fragment thereof which has human R-sopndin 2 activity;
viii) a DNA of R-spondin 2 variant having the nucleotide sequence encoding the amino acid sequence derived from the amino acid sequence represented by 22nd amino acid to 206th amino acid of SEQ ID NO:13 by deletion, substitution, or addition of 1 to 5 amino acids and has R-spondin 3 activity, or a fragment thereof which has human R-sopndin 2 activity;
ix) a DNA of R-spondin 3 having the nucleotide sequence encoding the amino acid sequence represented by 22nd amino acid to 272nd amino acid of SEQ ID NO:15 or a fragment thereof which has human R-sopndin 3 activity;
x) a DNA of R-spondin 3 variant having the nucleotide sequence encoding the amino acid sequence derived from the amino acid sequence represented by 22nd amino acid to 272nd amino acid of SEQ ID NO:15 by deletion, substitution, or addition of 1 to 5 amino acids and has R-spondin 3 activity, or a fragment thereof which has human R-sopndin 3 activity;
xi) a DNA of R-spondin 4 having the nucleotide sequence encoding the amino acid sequence represented by SEQ ID NO:17 or a fragment thereof which has human R-spondin 4 activity; and
xii) a DNA of R-spondin 4 variant having the nucleotide sequence encoding the amino acid sequence derived from the amino acid sequence represented by SEQ ID NO:17 by deletion, substitution, or addition of 1 to 5 amino acids and has R-spondin 4 activity, or a fragment thereof which has human R-spondin 4 activity.

12. The method according to claim 10, wherein the tumor is any one tumor selected from the group consisting of colon cancer, colorectal cancer, lung cancer, breast cancer, brain tumor, malignant melanoma, renal cell carcinoma, bladder cancer, leukemia, lymphomas, T cell lymphomas, multiple myeloma, gastric cancer, pancreas cancer, cervical cancer, endometrial carcinoma, ovarian cancer, esophageal cancer, liver cancer, head and neck squamous cell carcinoma, cutaneous cancer, urinary tract carcinoma, prostate cancer, choriocarcinoma, pharyngeal cancer, laryngeal cancer, thecomatosis, androblastoma, endometrium hyperplasy, endometriosis, embryoma, fibrosarcoma, Kaposi's sarcoma, hemangioma, cavernous hemangioma, angioblastoma, retinoblastoma, astrocytoma, neurofibroma, oligodendroglioma, medulloblastoma, ganglioneuroblastoma, glioma, rhabdomyosarcoma, hamartoblastoma, osteogenic sarcoma, leiomyosarcoma, thyroid sarcoma, Wilms tumor.

13. The method according to claim 11, wherein the tumor is any one tumor selected from the group consisting of colon cancer, colorectal cancer, lung cancer, breast cancer, brain tumor, malignant melanoma, renal cell carcinoma, bladder cancer, leukemia, lymphomas, T cell lymphomas, multiple myeloma, gastric cancer, pancreas cancer, cervical cancer, endometrial carcinoma, ovarian cancer, esophageal cancer, liver cancer, head and neck squamous cell carcinoma, cutaneous cancer, urinary tract carcinoma, prostate cancer, choriocarcinoma, pharyngeal cancer, laryngeal cancer, thecomatosis, androblastoma, endometrium hyperplasy, endometriosis, embryoma, fibrosarcoma, Kaposi's sarcoma, hemangioma, cavernous hemangioma, angioblastoma, retinoblastoma, astrocytoma, neurofibroma, oligodendroglioma, medulloblastoma, ganglioneuroblastoma, glioma, rhabdomyosarcoma, hamartoblastoma, osteogenic sarcoma, leiomyosarcoma, thyroid sarcoma, Wilms tumor.


SEQUENCE LISTING (17 sequences)

SEQ ID NO: 1 - DNA, 2339 bp, Homo sapiens
cgggtcgacgatttcgtcgcgccctcgcccctcccgggcctgcccccgtcgcgactggcagcacgaagctgagattgtggtttcctggtgattcaggtgggagtgggccagaagatcaccgctggcaaggactggtgtttgtcaactgtaaggactcatggaacagatctaccagggattctcagaccttagtttgagaaatgctgcaattaaaggcaaatcctatcactctgagtgatcgctttggtgtcgaggcaatcaaccataaagataaatgcaaatatggaaattgcataacagtactcagtattaaggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacacagacacaagacacacacgctcaaacccctgtccacatatacaaccatacatacttgcacatgtgtgttcatgtacacacgcagacacagacaccacacacacacatacacacacacacacacacgcacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtggcctgggaagaaaccttcttcccatcctgttcagcactcccagctgtgtgactttatcgttggagagtattgttaccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgataatctcaggtctcattccagacctggagagccatgagtctaaattttaagattcctgatgattggcatgttacccaaatttgagaagtgct

SEQ ID NO: 2 - DNA, 789 bp, Homo sapiens
atgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcc

SEQ ID NO: 3 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 4 - DNA, 927 bp, Artificial Sequence
  Description of Artificial Sequence GIPF V5His tag
atgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgccaagggcaattctgcagatatccagcacagtggcggccgctcgagtctagagggcccgcggttcgaaggtaagcctatccctaaccctctcctcggtctcgattctacgcgtaccggtcatcatcaccatcaccattga

SEQ ID NO: 5 - protein, 308 aa, Artificial Sequence
  Description of Artificial Sequence GIPF V5His tag
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAKGNSADIQHSGGRSSLEGPRFEGKPIPNPLLGLDSTRTGHHHHHH

SEQ ID NO: 6 - protein, 243 aa, Homo sapiens
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 7 - protein, 232 aa, Homo sapiens
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 8 - DNA, 23 bp, Artificial Sequence
  Description of Artificial Sequence forward primer
caccatgcggcttgggctgtctc

SEQ ID NO: 9 - DNA, 23 bp, Artificial Sequence
  Description of Artificial Sequence reverse primer
ggcaggccctgcagatgtgagtg

SEQ ID NO: 10 - DNA, 34 bp, Artificial Sequence
  Description of Artificial Sequence Synthetic DNA
acgcgtcgacccacatgcggcttgggctgtgtgt

SEQ ID NO: 11 - DNA, 30 bp, Artificial Sequence
  Description of Artificial Sequence Synthetic DNA
acgcgtcgacgtcgacctaggcaggccctg

SEQ ID NO: 12 - DNA, 621 bp, Homo sapiens
atggagacagacacactcctgttatgggtactgctgctctgggttccaggttccactggtgaccaaggcaaccgatggagacgcagtaagcgagctagttatgtatcaaatcccatttgcaagggttgtttgtcttgttcaaaggacaatgggtgtagccgatgtcaacagaagttgttcttcttccttcgaagagaagggatgcgccagtatggagagtgcctgcattcctgcccatccgggtactatggacaccgagccccagatatgaacagatgtgcaagatgcagaatagaaaactgtgattcttgctttagcaaagacttttgtaccaagtgcaaagtaggcttttatttgcatagaggccgttgctttgatgaatgtccagatggttttgcaccattagaagaaaccatggaatgtgtggaaggatgtgaagttggtcattggagcgaatggggaacttgtagcagaaataatcgcacatgtggatttaaatggggtctggaaaccagaacacggcaaattgttaaaaagccagtgaaagacacaatactgtgtccaaccattgctgaatccaggagatgcaagatgacaatgaggcattgtccaggagggtaa

SEQ ID NO: 13 - protein, 206 aa, Homo sapiens
METDTLLLWVLLLWVPGSTGDQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGG

SEQ ID NO: 14 - DNA, 819 bp, Homo sapiens
atggagacagacacactcctgttatgggtactgctgctctgggttccaggttccactggtgaccaaaacgcctcccggggaaggcgccagcgaagaatgcatcctaacgttagtcaaggctgccaaggaggctgtgcaacatgctcagattacaatggatgtttgtcatgtaagcccagactattttttgctctggaaagaattggcatgaagcagattggagtatgtctctcttcatgtccaagtggatattatggaactcgatatccagatataaataagtgtacaaaatgcaaagctgactgtgatacctgtttcaacaaaaatttctgcacaaaatgtaaaagtggattttacttacaccttggaaagtgccttgacaattgcccagaagggttggaagccaacaaccatactatggagtgtgtcagtattgtgcactgtgaggtcagtgaatggaatccttggagtccatgcacgaagaagggaaaaacatgtggcttcaaaagagggactgaaacacgggtccgagaaataatacagcatccttcagcaaagggtaacctatgtcccccaacaaatgagacaagaaagtgtacagtgcaaaggaagaagtgtcagaagggagaacgaggaaaaaaaggaagggagaggaaaagaaaaaaacctaataaaggagaaagtaaagaagcaatacctgacagcaaaagtctggaatccagcaaagaaatcccagagcaacgagaaaacaaacagcagcagaagaagcgaaaagtccaagataaacagaaatcggtatcagtcagcactgtacactag

SEQ ID NO: 15 - protein, 272 aa, Homo sapiens
METDTLLLWVLLLWVPGSTGDQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 16 - DNA, 705 bp, Homo sapiens
atgcgggcgccactctgcctgctcctgctcgtcgcccacgccgtggacatgctcgccctgaaccgaaggaagaagcaagtgggcactggcctggggggcaactgcacaggctgtatcatctgctcagaggagaacggctgttccacctgccagcagaggctcttcctgttcatccgccgggaaggcatccgccagtacggcaagtgcctgcacgactgtccccctgggtacttcggcatccgcggccaggaggtcaacaggtgcaaaaaatgtggggccacttgtgagagctgcttcagccaggacttctgcatccggtgcaagaggcagttttacttgtacaaggggaagtgtctgcccacctgcccgccgggcactttggcccaccagaacacacgggagtgccagggggagtgtgaactgggtccctggggcggctggagcccctgcacacacaatggaaagacctgcggctcggcttggggcctggagagccgggtacgagaggctggccgggctgggcatgaggaggcagccacctgccaggtgctttctgagtcaaggaaatgtcccatccagaggccctgcccaggagagaggagccccggccagaagaagggcaggaaggaccggcgcccacgcaaggacaggaagctggaccgcaggctggacgtgaggccgcgccagcccggcctgcagccctga

SEQ ID NO: 17 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP